(19) United States
(12) Patent Application Publication
(10) Pub. No.: US 2007/0244471 A1
(43) Pub. Date: Oct. 18, 2007

(54) SYSTEM AND METHOD FOR MANAGING THE OPERATION OF A BATTERY POWERED SURGICAL TOOL AND THE BATTERY USED TO POWER THE TOOL

(76) Inventor: Don Malackowski, Schoolcraft, MI (US)

Correspondence Address: INTEL. PROP./ RND;STRYKER CORPORATION, 4100 EAST MILHAM AVE., KALMAZOO, MI, 49001-6197, US

(21) Appl. No.: 11/739,952

(22) Filed: Apr. 25, 2007

Related U.S. Application Data
(63) Continuation-in-part of application No. 11/551,335, filed on Oct. 20, 2006.
(60) Provisional application No. 60/729,338, filed on Oct. 21, 2005.

(51) Int. Cl. A61B 17/00; H02J 7/00

(52) U.S. Cl. 606/1; 320/107

(57) ABSTRACT

A surgical power tool system including a cordless powered surgical tool, a battery for energizing the tool and a charger for charging the battery. The battery includes a memory. When the battery is attached to the tool, a processor integral with the tool writes data to the memory regarding the operation of the tool. When the battery is attached to the charger, the data are read out the battery memory. These data are then evaluated to determine the operational state of the tool.

### RELATIONSHIP TO EARLIER FILED APPLICATIONS

This application is a continuation-in-part based on U.S. patent application Ser. No. 11/551,335 filed 20 Oct. 2006 which claims priority under 35 U.S.C. Sec 119 from U.S. Provisional Patent App. No. 60/729,338 filed 21 Oct. 2005.

### FIELD OF THE INVENTION

This invention is related to battery powered surgical tools. More particularly, this invention is related to system for managing these tools and the batteries used to power them, based on data contained in the batteries

### BACKGROUND OF THE INVENTION

A battery often energizes a powered surgical tool used in an operating room to perform a surgical procedure. The use of a battery eliminates the need to provide a power cord connected to an external power source. The elimination of the power cord offers several benefits over corded surgical tools. Surgical personnel using this type of tool do not have to concern themselves with either sterilizing a cord so that it can be brought into the sterile surgical field surrounding the patient or ensuring that, during surgery, an unsterilized cord is not inadvertently introduced into the surgical field. Moreover, the elimination of the cord results in the like elimination of the physical clutter and field-of-view blockage the cord otherwise brings to a surgical procedure.

In an operating room, batteries are used to power more than the tools used to perform the surgical procedure. Batteries are also used to energize the power consuming components integral with a personal protection system surgical personnel sometimes wear when performing a procedure. This system typically includes some type of hooded garment. Internal to the garment is a ventilation unit for circulating air within the garment. Some of these systems also have lights for illuminating the surgical site or radios that facilitate conventional spoken level conversation with other persons involved in performing the procedure. Each of these units, the ventilation unit, the light unit and the radio, requires a source of power. By providing this power from the battery, the need to attach cords to each individual wearing such a unit is eliminated. This, in turn, reduces number of cords in the operating room persons would otherwise have to avoid. Further, eliminating these cords likewise eliminates the restrictions of movement they place on the individual using the system.

An integral part of any battery-powered device is, naturally, the battery. Most battery-powered surgical devices used in an operating room are designed to be used with rechargeable batteries. These rechargeable batteries typically include one or more NiCd cells. Once a battery is discharged, it is coupled to a complementary charger. The charger applies a current to the battery's cells to store energy in the cells.

Unlike other rechargeable batteries, a rechargeable battery intended for use with a surgical tool must be sterilizable so that it can be placed in close proximity to the open surgical site on a patient. Often, these batteries are sterilized by placing them in an autoclave wherein the atmosphere is saturated with water vapor (steam), the temperature is approximately 270° F. (132° C.) and the atmospheric pressure is approximately 30 psi (Gage) (1552 mmHg). The repetitive exposure to this environment causes a battery cells' ability to store electric charge to degrade. Often this is referred to as degradation in the “state of health” of the battery.

The Applicant's U.S. Pat. No. 6,018,227, BATTERY CHARGER ESPECIALLY USEFUL WITH STERILIZABLE RECHARGEABLE BATTERY PACKS, issued Jan. 25, 2000 and incorporated herein by reference, discloses a means to determine the voltage at load of a battery. Inferentially, this is a measure of the internal resistance of the battery. Unfortunately, this information alone does not provide a complete measure of the battery state of health. For example, this information alone does not provide information if the stored energy is sufficient to power the device to which the battery is attached for the time required to perform the surgical procedure. This means that, during the performance of a procedure, if the battery's stored energy appreciably depletes, the procedure is interrupted to replace the battery. This increases the overall time takes to perform the procedure. This interruption runs contrary to one of the goals of modern surgery which is to perform the procedure as quickly as possible to lessen the time the patient's internal organs are exposed, and therefore open to infection, and the amount of time a patient is held under anesthesia.

A corded power tool does offer one appreciable advantage over its battery powered equivalent. A corded surgical power tool typically provides one or more feedback signals to the console that supplies the current to tool. Some feedback signals are explicitly designed as such. For example, sometimes a powered surgical tool includes a temperature sensor. A signal representative of sensed tool temperature is feedback to the console. Some feedback signals are inherently supplied to the console as a result of the energization of the tool power consuming unit. For example, the energization signal functions as a feedback signal in that it indicates the power drawn by the tool power consuming unit. The states of the feedback signals are monitored by the control console. Thus, for example, the console monitors the temperature sensor to determine if the tool temperature exceeds a set level. The console can monitor the energization signal to determine if the current (power) drawn by the tool likewise exceeds a set level. If this monitoring indicates that the tool appears to be in, or approaching, an out of boundary condition, i.e., excessively high temperature or excessive current draw, the console can take appropriate action. This action includes generating a warning message indicating the tool is in/approaching and out of boundary condition. Alternatively, when the tool is in/approaches the out of boundary condition, the console can inhibit to the level of totally negating the assertion of the energization signal the console. A console may be configured to take this action if the entry of the tool into the out of boundary state could result in the potential of injury.

A batter powered tool is, of course, not connected to a control console. There is no ready means to determine if the tool is in/approaching an outer of boundary state that can result in tool malfunction. This means that sometimes battery powered surgical tools are operated until such malfunction occurs. Depending on the type of malfunction, this may mean interrupting the procedure until a substitute, properly functioning, tool is available. Having to so delay the procedure can length the time the patient's tissue is exposed to the ambient environment and, therefore, open to infection. Having to so delay a surgical procedure also serves to lengthen the time the time the patient has to be held under anesthesia. This is contrary to goal of modern surgery which is that it preferable to as reasonably as possible, hold the amount of time the patient is kept under anesthesia to a minimum.

One can provide the data from these devices through wireless communications systems. One system is disclosed in the Applicant's U.S. Patent Application No. 60/694,592, POWERED SURGICAL TOOL WITH SEALED CONTROL MODULE, filed 28 Jun. 2005, U.S. Patent Publication No. 20070085496 A1, now U.S. Pat. No. ______, incorporated herein by reference. A disadvantage of the above-mentioned system is that it requires the addition of a wireless communications system into the operating room. The expense of providing such a system limits the locations where they are installed.

The Applicant's Assignee's U.S. Pat. No. 5,977,746, RECHARGEABLE BATTERY PACK AND METHOD FOR MANUFACTURING SAME, issued 2 Nov. 1999 and incorporated herein by reference, discloses a rechargeable battery especially designed to withstand the rigors of autoclave sterilization. The battery of this invention includes a cluster of cells that are bound together by top and bottom plastic binders. Conductive straps extending between openings formed in the binders connect the cells. One of the straps is a fuse that opens upon a more than a specific current flowing through it. More specifically, the current through the fuse heats the material forming the fuse so a section of the fuse vaporizes. This vaporization of the fuse section separates the rest of the fuse into two sections.

The above battery pack has proven useful for storing the charge needed to energize a cordless surgical tool. However, the cells internal to the battery pack can generate significant amounts of heat. This causes the temperature of the cells to rise. Sometimes, the temperature rise between the cells is uneven. This uneven thermal loading of cells can result in an electrical imbalance of the cells. If the cells become so imbalanced, both the immediate utility of the battery to supply energy at a particular time and its useful lifetime may diminish.

### SUMMARY OF THE INVENTION

This invention relates to a new and useful battery operated surgical tool system. The battery of this system is designed for use in a harsh environment such as in a hospital where the battery is autoclave sterilized. The battery and battery charging system of this invention are further designed to record and transmit data about the surgical tools the battery is used to energize.

The battery of this invention includes a set of rechargeable cells. Also internal to the battery are a data recording unit and a temperature sensor. Both the data recording unit and temperature sensor are powered by the battery cells so that they are always on, regardless of whether or not the battery is being used to power a device or is being charged. Collectively, the data recording unit and temperature sensor are configured to record data about the temperature of the battery.

The battery charger of this invention includes a current source for charging the battery. Also internal to the battery charger is a processor and a load resistor. The processor regulates the actuation of the current source and connection of the battery to the load resistor.

The processor also reads the data stored in the battery data recording unit. Depending on the data indicating the history of the battery, the processor may conduct a state of health evaluation of the battery. For example, a state of health evaluation may be performed if the data in the data recording unit indicates that battery was continually at a temperature above a threshold level for more than a given period of time. To perform a state of health evaluation, the processor both measures the voltage-at-load of the battery and the quantity of energy input to the battery. Often, this last evaluation is made by first fully discharging the battery. The results of the state of health evaluation are displayed.

Another feature of this invention is that, while the battery is being used to power a device, the device writes data into the data recording unit. When the battery is attached to the charger, the data recording unit writes out the stored device data to the charger processor. The charger processor, in turn, forwards these data to another device. Thus, information about the operating state of a battery powered device is available to persons charged with maintaining the device. This information is available even though there is no corded link or RF/IR/ultrasonic wireless communications link to the device.

Often these data are made available through a communications bus to a device monitor, also part of the system of this invention. The device monitor includes a processor. The device monitor processor evaluates the received data to determine the operating states of both the tool and the battery. If these evaluations indicated that the tool or battery is approaching a failure condition, the device monitor informs the personnel responsible for maintaining the tool/battery of this condition. This allows for maintenance or replacement of the tool/battery before it can fail during the time when such replacement could affect a medical/surgical procedure.

### BRIEF DESCRIPTION OF THE DRAWINGS

The invention is pointed out with particularity the in the claims. The above and further features and benefits of the battery, battery charger and method for charging a battery of this invention may be better understood from the following detailed description taken in conjunction with the accompanying drawings in which:

FIG. 1 is a perspective view of a battery and battery charger of this invention;

FIG. 2 is a perspective view of the battery;

FIG. 3 is an exploded view of the battery of this invention;

FIG. 4 is a perspective view of the battery housing;

FIG. 5 is a cross sectional view of the battery housing;

FIG. 5A is an enlarged cross sectional view of the top edge of the battery housing;

FIG. 6 is an exploded view of the cell cluster internal to the battery;

FIG. 7 is an exploded view of the binder assembly, here the top binder assembly, of the cell cluster;

FIG. 8 is a plan view of the thermal fuse internal to the top binder assembly;

FIG. 9 is a cross sectional view of the battery lid;

FIG. 10 is a plan view of the undersurface of the battery lid;

FIG. 11 is an enlarged cross sectional view of the bottom lip of the battery lid;

FIG. 12 is a schematic drawing of the electrical components internal to the battery;

FIG. 13 is a block diagram of some of the sub circuits internal to the battery microcontroller;

FIG. 14 depicts some to types of data stored in the memory integral with the battery microcontroller;

FIG. 15A is a plan view illustrating one of the fixtures in which the components forming the cell cluster are placed in order to facilitate assembly of the cluster;

FIG. 15B is side view illustrating how the components forming the cell cluster are fitted in a pair of fixtures;

FIG. 16 is a diagrammatic illustration of the welding process used to complete the assembly of the cell cluster

FIG. 17 is a cross sectional view of the interface of the battery housing and battery lid prior to the welding of these components together;

FIG. 18 is diagrammatic representation of how the battery housing and lid are welded together;

FIG. 19 is a cross sectional view of the interface of the battery housing and battery lid after the welding process;

FIG. 20 is an exploded view of relationship of the charger base to the charger housing;

FIG. 20A is a perspective view of how the discharger resistors and complementary heat sink are secured to the charger base;

FIG. 21 is a cross sectional view of some of the components internal to the charger;

FIG. 22 is a block diagram of sub-circuits internal to the charger and a module attached to the charger;

FIGS. 23A and 23B collectively form a flow chart of the process steps performed by the battery microcontroller to monitor the autoclaving of the battery;

FIGS. 24A, 24B and 24C collectively form a flow chart of the process steps executed by the charger in order to charge a batter according to the process of this invention;

FIG. 25 is a flow chart of the process steps executed by the processor internal the charger to ensure that the charger temperature does not rise to potentially unsafe levels;

FIG. 26 is a block diagram illustrating of the tool communications system of this wherein the battery and charger are used to facilitate the exchange of data between the surgical tool and other components;

FIG. 27 is a block diagram of the components of tool of the system of this invention;

FIG. 28 is a block diagram of data stored in the tool history file internal to the battery microcontroller;

FIG. 29 is a flow diagram of the process steps executed in the tool communication system of this invention.

FIGS. 30A, 30B and 30C collectively form a flow chart of the steps executed by the device monitor to evaluate the battery and tool of the system of this invention;

FIG. 31 is the contents of a file maintained by the device monitor in order to evaluate whether or not a battery of the system of this invention is subjected to excessive use;

FIG. 32 is a block diagram of a tool log the device monitor maintains for a tool of the system of this invention;

FIG. 33 is a block diagram of an upload data file the system of this invention loads into a tool; and

FIG. 34 is a flow chart of the process by which new tool operating instructions are uploaded into the tool.

### DETAILED DESCRIPTION

### I. Overview

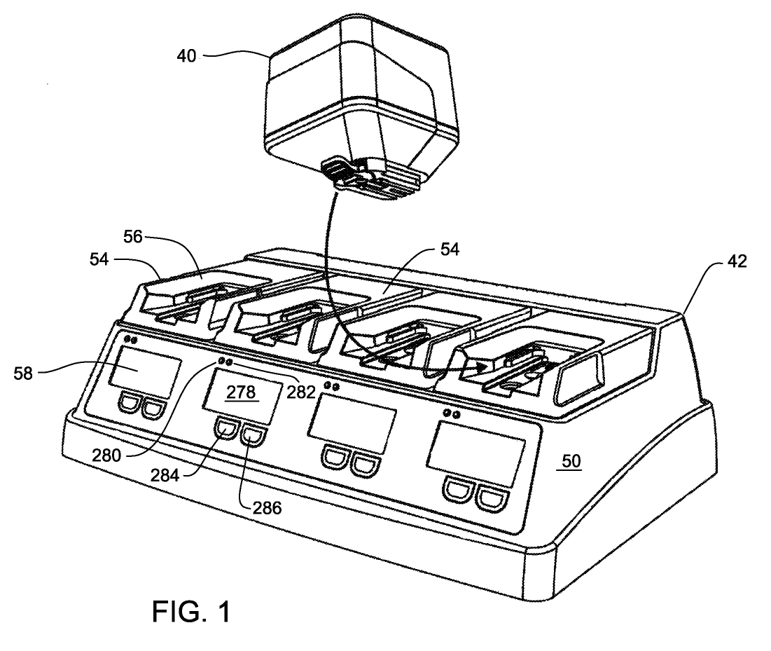

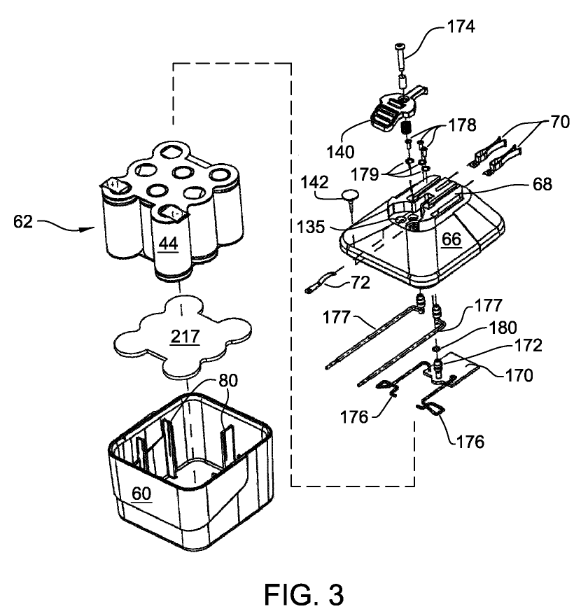

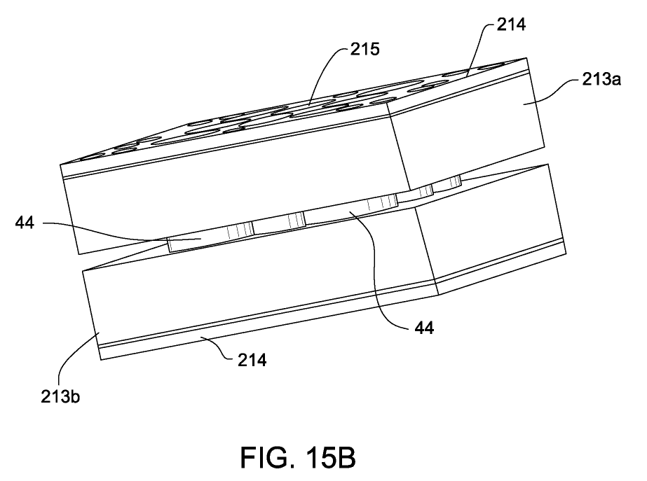

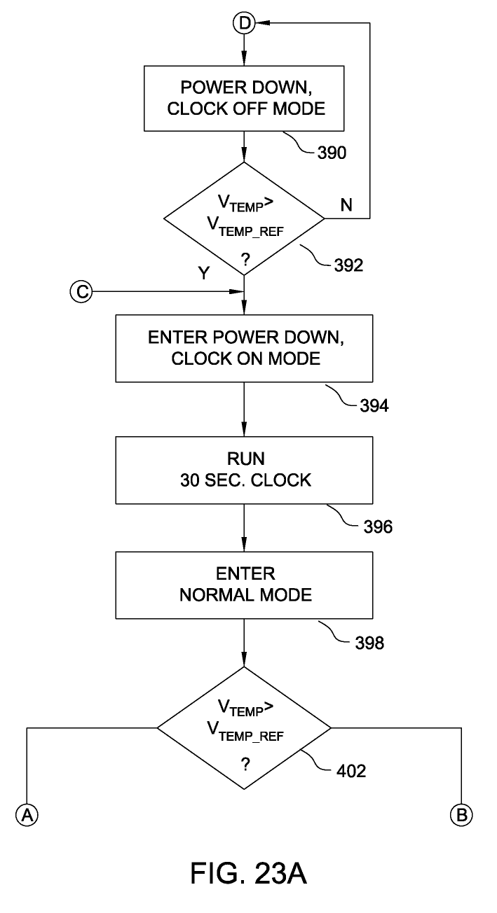

FIG. 1 illustrates a battery 40 and battery charger 42 constructed in accordance with this invention. Battery 40, includes a set of rechargeable cells 44 (FIG. 3) a microcontroller 46 and a temperature sensor 48 (FIG. 12). Battery charger 42 includes a housing 50 with a number of pockets 52 (FIG. 20). Each pocket 52 removably receives a module 54 associated with a specific type of battery. The module 54 is shaped to define a complementary socket 56 for receiving the head end of the associated battery 40. Internal to the battery charger 42 are components for reading the data stored in the battery microcontroller 46 and for charging the battery cells 44. A plurality of I/O units 58 are attached to the charger 42. Each I/O unit 58 functions as the sub-assembly through which instructions are entered and charge state information presented about an individual one of the batteries 40 attached to the charger 42.

### II. Battery And Method of Battery Assembly

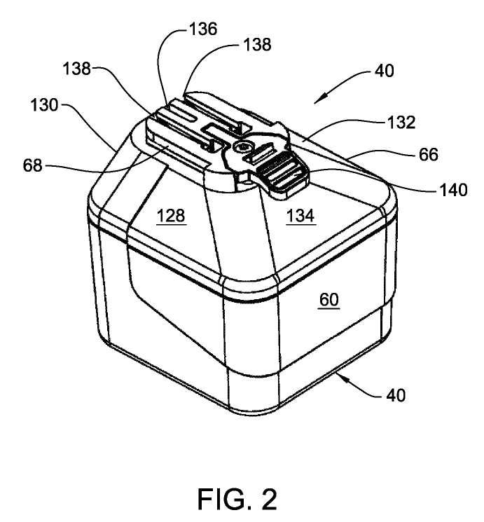

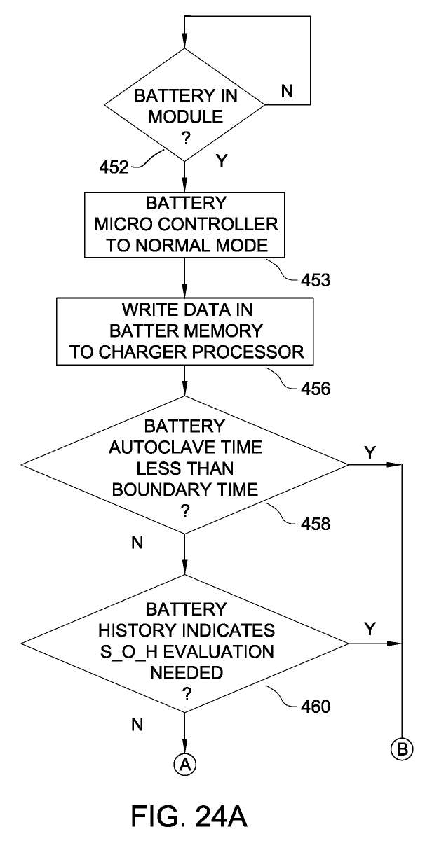

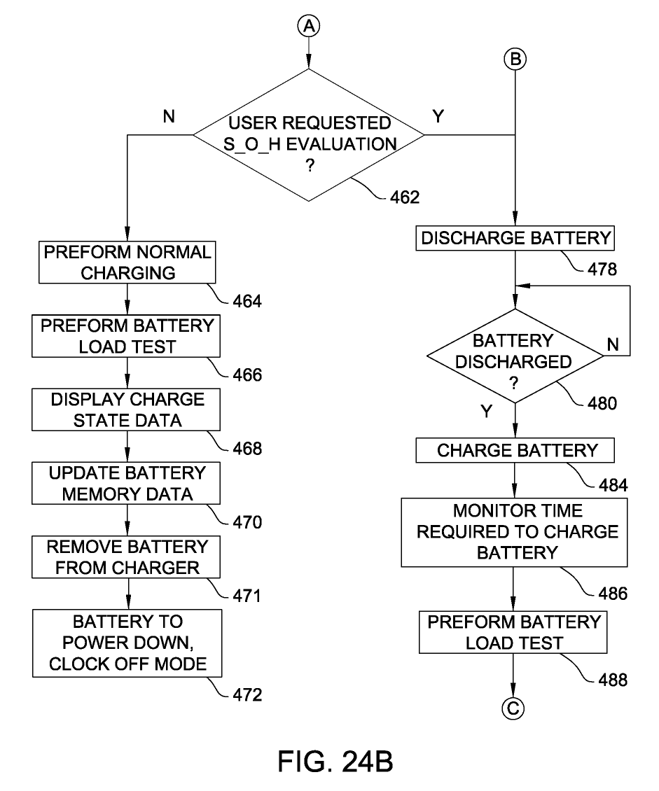

As seen in FIGS. 2 and 3, a battery 40 of this invention includes a housing 60. Rechargeable cells 44 are arranged in a cluster 62 seating in housing 60. A lid 66 is sealing disposed over the open top end of the housing 60. Lid 66 is formed with a head 68. The lid 66 is the battery structural component to which the microcontroller 46 and temperature sensor 48 are mounted. In the illustrated version of the invention, the lid head 68 is dimensioned to fit into a complementary socket formed in the power tool 522 (FIG. 22) the battery 40 is intended to power. The lid head 68 is provided with two contacts 70 and a single contact 72. Contacts 70 are the conductive members through which the charger 42 applies a charging current to the cells 44 and from which the power tool 522 (FIG. 23) draws an energizing current. Contact 72 is the contact through which data and instructions are written into and read out from the microcontroller 46. Thus, data are exchanged between the charger 42 and battery microcontroller 46 using a one-wire signal exchange protocol. One such protocol is the Dallas Semiconductor One-Wire protocol.

Battery housing 60 is formed from a single piece of plastic that is transmissive to light energy emitted at 980 nanometers. By “transmissive” it is understood the plastic is at least “partially” transmissive. In most versions of the invention the plastic is at least 55% percent transmissive. In more preferred versions, the plastic is at least 75% transmissive. In one version of the invention, housing 60 is formed from a polyphenylsulfone plastic. One such plastic from which housing 60 is formed is sold under the brand name RADEL by Solvay Advanced Polymers, of Alpharetta, Ga., United States. This plastic is partially transparent. For aesthetic reasons, the plastic forming housing 60 may be dyed to be opaque at visible wavelengths. If housing 60 is so dyed, the dye should be selected so that it does not appreciably interfere with transmissivity of photonic energy at the 980 nanometer range. As discussed below this is the wavelength at which, in one process lid 66 is laser welded to housing 60.

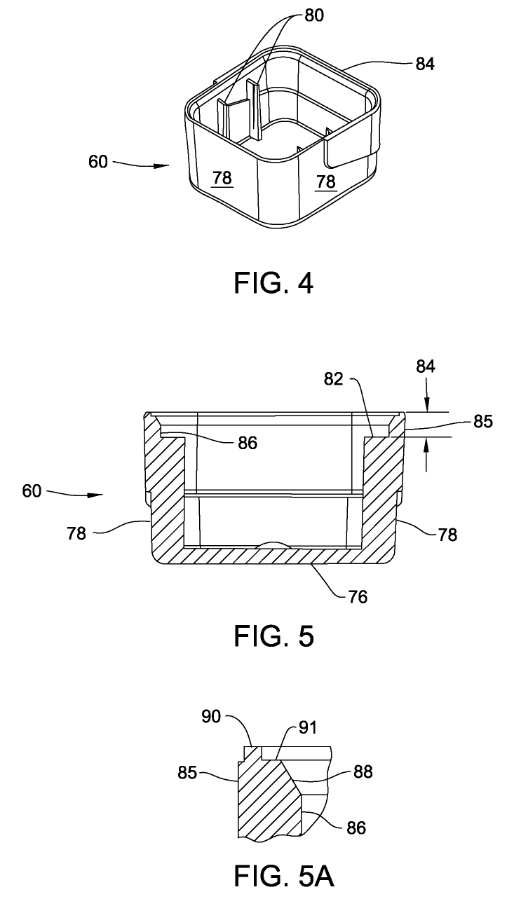

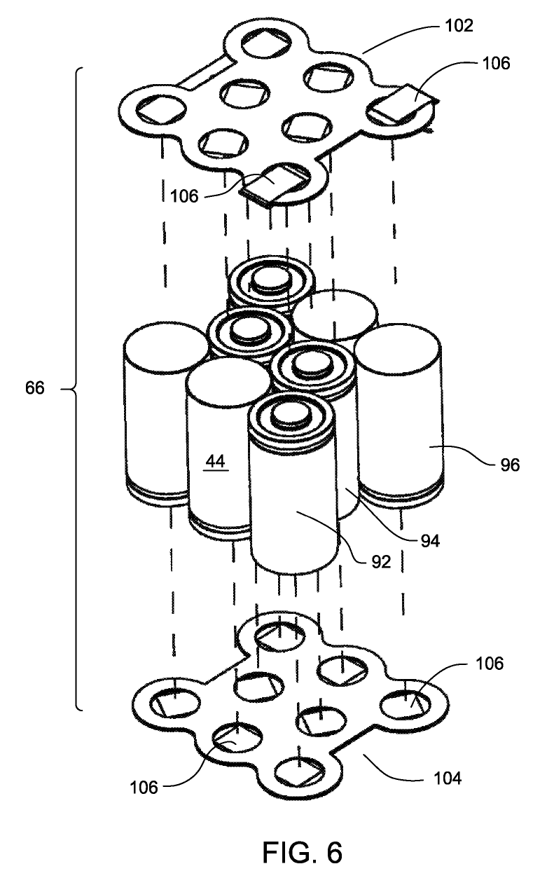

As seen in FIGS. 4, 5 and 5A, housing 60 is formed to have a generally rectangular base 76. Four interconnected walls 78 extend upwardly from the perimeter edges of the base 76. For aesthetic reasons, the corners of the base 76 and the corners where walls 78 abut are rounded. Housing 60 is further shaped so that walls 78 taper outwardly away from base 76. The housing 60 is further formed so that ribs 80 extend inwardly from the inner surfaces of the walls 78 from the top surface of the base 76. Each wall 78 may be formed with one, two or more ribs 80. Ribs 80 provide structural rigidity to the walls and minimize movement of the cell cluster 62 within the housing 60.

Each housing wall 78 has an inner vertical surface 86. (In the cross sectional view of FIG. 5 rib 50 is seen below the top of the inner surface 86.) Above the inner vertical surface 86 there is a tapered face 88 that angled outwardly relative to the vertical surface 86. A reveal 90 forms the top most portion of each lip 78. The reveal 90 has a generally square cross sectional profile. The width of the reveal 90 is less than that of the vertical surface 91 that extends between the top edge of the lip outer surface 85 and the top edge of tapered inner face 88. Housing 60 is thus formed so that reveal 90 is located inwardly of both the top edge of the lip outer surface and the top edge of the tapered inner face 88.

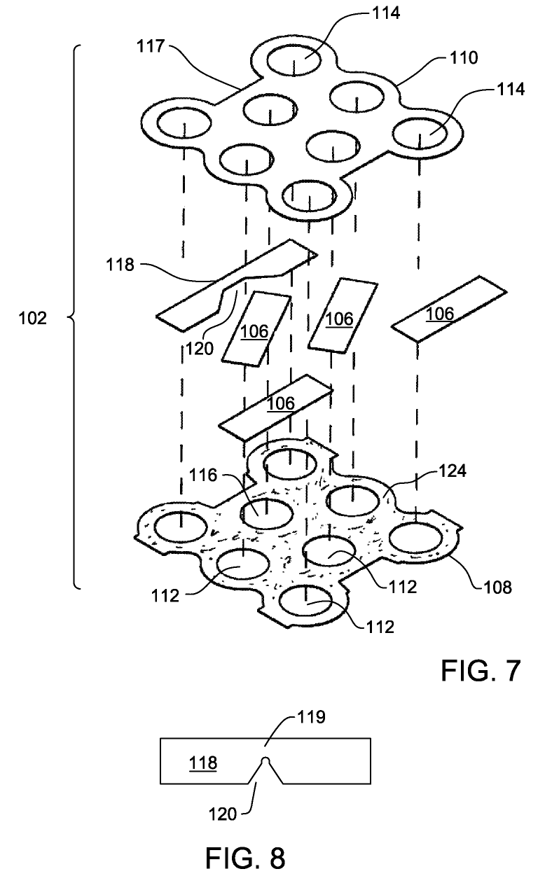

As seen by reference to FIG. 6, the cell cluster 62 includes a plurality of rechargeable cells 44. As is known from the above-identified, incorporated herein by reference U.S. Pat. No. 5,977,746, the outer cylindrical surface of each cell 44, which functions as the cell ground, is covered with polyimide tape, (not shown).

Cells 44 are arranged in a three abutting rows 92, 94 and 96, such that the cells in one row abut the cells in the adjacent row. In each row 92-94, the adjacent cells 44 abut. The cells 44 are arranged so that there are three cells in the outer rows, rows 92 and 96, and two cells in the center row, row 94. This arrangement ensures that each cell has an outer perimeter section of at least 10% and, more preferably at least 20%, that neither abuts an adjacent cell nor is concealed behind an adjacent row of cells. Thus a perimeter section of at least 10%, and more preferably at least 20%, of each cell 44 forms a portion of the outer perimeter of the array of cells forming the cell cluster 62.

The top and bottom orientation, the orientations of, respectively, the positive and negative terminals, of the cells 44 is arranged as a function to the extent the cells are to be connected together in a series or parallel arrangement in order to provide a charge at a particular voltage level and current.

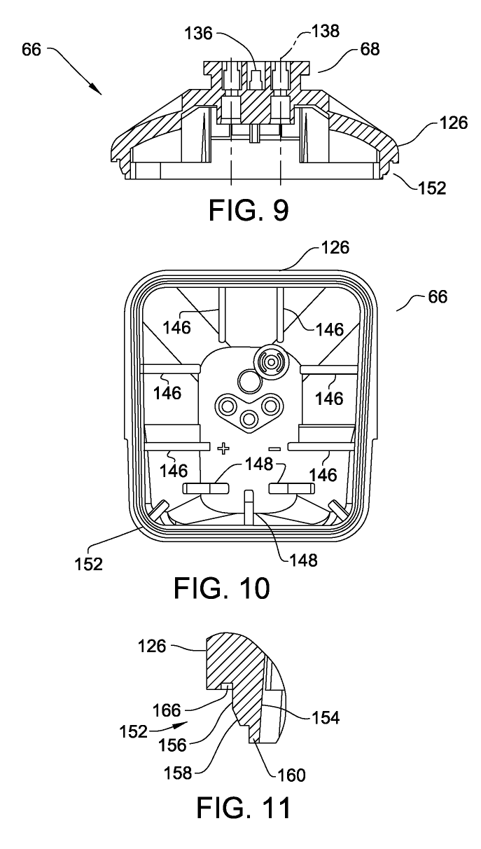

The cells 44 are held together to form the cluster 62 by top and bottom binder assemblies 102 and 104, respectively. Each binder assembly 102 and 104 includes a number of conductive straps 106 that are in the form of thin strips of metal. As seen in FIG. 7, which shows the top binder assembly 102, each binder assembly includes inner and outer binders 108 and 110, respectively. (For reference, the “inner” binder is understood to be the binder closest to the cells 44; the “outer” binder is spaced from the cells.) Each binder 108 and 110 is formed from a flexible plastic material such as a polyester sold under the trademark MYLAR by DuPont. Each binder 108 and 110 is formed with a number of openings 112 and 114, respectively. Binders 108 and 110 forming the upper binder assembly 102 are further formed so as to define along the outer perimeter thereof aligned notches 116 and 117, respectively.

Conductive straps 106 are sandwiched between the binders 108 and 110. Each conductive strap 106 is positioned to have one end that extends into the space subtended by aligned pair of binder openings 112 and 114. Some conductive straps 106 are positioned so that that the second ends of the straps extend into one of the aligned pairs of binder openings 112 and 114. These conductive straps 106 electrically connect the terminals of adjacent cells 44. Two of the conductive straps 106 are positioned so that their second ends project beyond the perimeters of the binders 108 and 110. These two conductive straps 106, seen in FIG. 6, function as the members that provide electrical connections between the cell cluster 62 and the contacts 70.

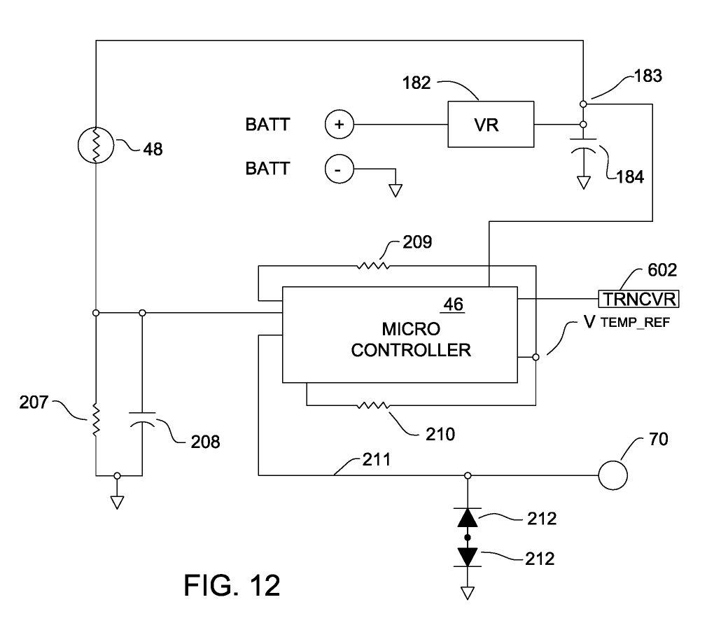

A fuse 118 is also disposed between the binders 108 and 110 forming top binder assembly 102. The fuse 118, best seen in FIG. 8, is formed of a conductive metal that when the current flow therethrough causes material heating to the point the metal vaporizes. In one version of the invention, fuse 118 is formed from nickel or a nickel alloy. Fuse 118 is generally in the form of a planar strip. The fuse 118 is further formed so as have notch 120 that extends inwardly from the one of the longitudinal side edges of the metal strip forming the fuse. (The geometries of notch 120 of the fuse of FIG. 7 and of the fuse 118 of FIG. 8 are slightly different.) In FIG. 8, section 119 of fuse 118, the narrowest width section, defines the widest portion of notch 120.

A binder assembly 102 or 104 of this invention is assembled by first placing one of the binders 108 and 110 in a jig. More particularly, the jig is formed with a recess designed in which the binders 108 and 110 are designed to precisely seat. Extending into the recess from the base of the jig are spaced apart fingers. The fingers extend through into the spaces subtended by binder openings 112 and 114. The fingers are spaced so as to define spaces therebetween into which the conductive straps 106 and fuse 118 are seated.

The exposed surface of the binder 108 or 110 seated in the jig recess is provided with an adhesive. In some versions of the invention, the adhesive is pre-applied to the binder 108 or 110. At manufacture, a protective sheet that covers the adhesive is removed. In FIG. 7, the adhesive is represented as stippling 124 on inner binder 108.

Once the first binder 108 or 110 is set in the jig, the conductive straps 106 and fuse 118 are set over the binder. More specifically, the conductive straps 106 and fuse 118 are set between the fingers that extend through the binder openings 112 or 114. The second binder 110 or 108 is then disposed over the partially assembled unit. In some versions of the invention, adhesive material may also disposed over the surface of the second binder that abuts the first binder.

As a consequence of the assembly of the binders 108 and 110, each inner binder opening 112 is aligned with an associated one of the upper binder openings 114. Inner and outer binder notches 116 and 117, respectively, are also aligned. It should further be appreciated that, during the assembly of the binder assembly 102, fuse 118 is positioned so that fuse notch 120 is within the area where the binders 108 and 110 are sandwiched together. The portion of the fuse 118 that defines fuse notch 120 is within the space subtended by binder notches 116 and 117. In more preferred versions of the invention, the fuse is positioned so that the thinnest section of the fuse, the portion defining the widest section of fuse notch 120, is spaced from the binders 108 and 110.

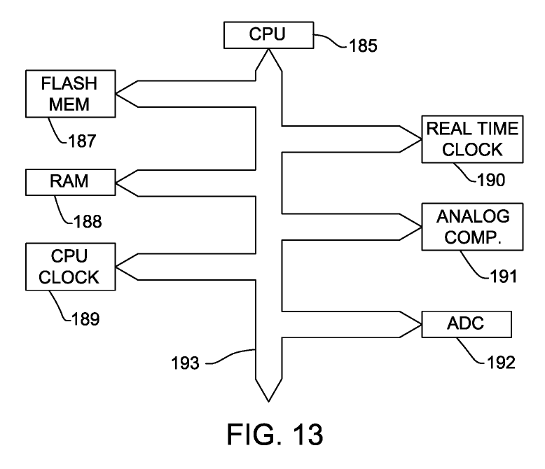

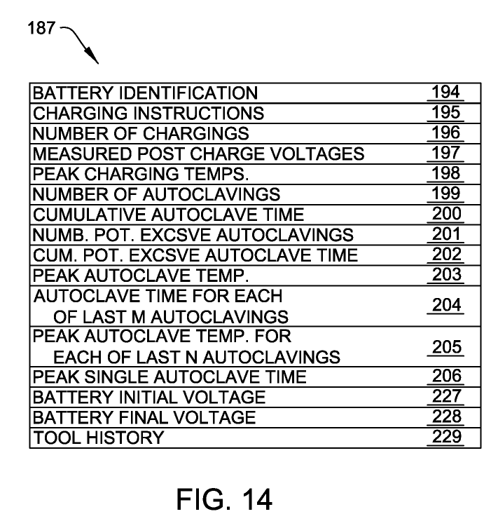

The battery lid 66 is now described by reference to FIGS. 2, 9, and 10. In one version of the invention, lid 66 is a single component formed from a polyphenelsulfone plastic such as the RADEL R plastic. For aesthetic reasons, the plastic forming the lid may be dyed to be opaque at the visible wavelengths. If the lid 66 is to be secured to the housing 60 by the below discussed laser welding process, the lid should be formed of material that absorbs the photonic energy at the wavelength emitted by the laser. The aesthetic dye can function as this material. Thus, in the described version of the invention, the dye absorbs energy emitted in the 980 nanometer range. Lid 66 is shaped to have a generally rectangular base 126 that has a geometry that subtends the top edges of the housing walls 78. Four panels 128, 130, 132 and 134 extend inwardly and upwardly from the sides of the base 126. The panels 128-134 meet at a planar horizontal surface 136 from which the battery head 68 upwardly projects. Panels 128 and 132 are the side panels and are symmetric relative to each other. Panel 130 is the front panel; panel 134 is the rear panel. Relative to the horizontal plane, front panel 130 has a steep upward slope; the slope of rear panel 134 is shallower.

Battery head 68 is formed to have a slot 136 and two slots 138. Each of slots 136 and 138 are open to the front face of the head 68. Slot 136 is centered along the longitudinal centerline of the battery 40. Slots 138 are parallel to and located on either side of slot 138. Contact 72, the contact through which signals are exchanged with microcontroller 46 extends into slot 136. Contacts 70, the contacts through which charge is stored in and drawn from cells 44, is disposed in slots 138.

A latch 140 is pivotally mounted to the battery head. The latch 140 holds the battery 40 to the power consuming device to which the battery is connected. A pressure relief valve 142 is mounted to horizontal surface under the latch 140. Not identified are the openings in which latch 140 and valve 142 are mounted and the assembly that pivotally holds the latch to the battery lid 66.

A number of ribs 146 and 148 extend inwardly from the inner surface of lid panels 128-134. The ribs 146 and 148 are generally rectangular in shape and extend into the inner surface of the lid below horizontal surface 135. Ribs 146 are relatively tall; ribs 148 are short. Two ribs 146 extend inwardly from panels 128, 132 and 134. A single rib 148 extends inwardly from front panel 130. An additional rib 148 extends inwardly from each of the side panels 128 and 132 immediately adjacent the front panel. Each rib 148 is further formed so that the outer end is downwardly stepped relative to the portion of the rib immediately adjacent the panel from which the rib extends. Ribs 146 and 148 minimize, if not completely block, vertical displacement of the cell cluster 62.

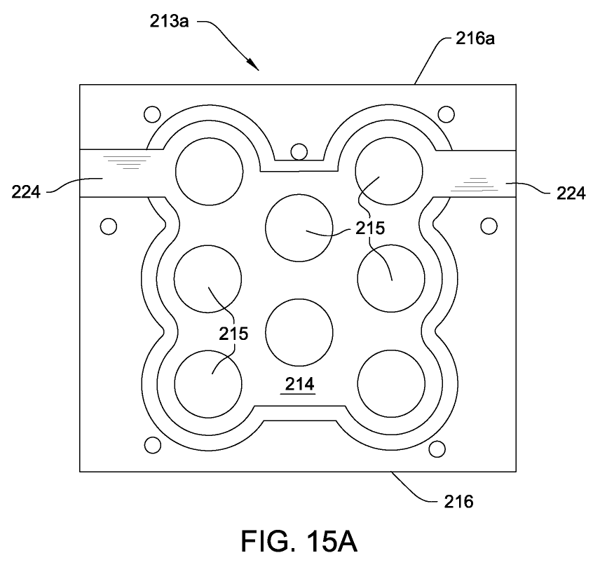

Battery lid 66 also has a lip 152 that extends downwardly from the base 126 around the perimeter of the lid. As seen best in FIG. 11, the lip 152 is located inwardly of the outer vertical surface of the base 126. Lid 66 is formed so that lip 152 has an inner vertical surface 154 that is flush with the adjacent inner surface of the base 126. The lip 152 has an outer vertical surface 156 located inward of the outer perimeter of the base 126. The lip 152 is further formed to have a tapered surface 158 that extends below vertical surface 154. Surface 158 tapers inward toward the center of the lid 66. A rectangularly shaped flange 160 forms the bottommost portion of lip 152 and, by extension, the bottommost structural feature of the battery lid 66. The bottommost portion of inner vertical surface 154 forms the inner surface of flange 160. A parallel vertical surface 164 that is inwardly stepped relative to the adjacent surface 158 forms the outer wall of the flange 160.

Battery lid 66 is further formed to define a rectangular notch 166 that extends upwardly from the bottom surface of base 126. The base 126 is formed so that notch 166 is located immediately in front of and is partially defined by lip outer vertical surface 156. In some versions of the invention, the notch is absent from the lid 66.

Returning to FIG. 3, it can be seen that a printed circuit board 170 is mounted in the battery lid 66. Printed circuit board 170 is the component to which battery microcontroller 46 and temperature sensor 48 are mounted (not illustrated). Circuit board 170 is fitted in the lid 66 to seat against the inwardly stepped edges of ribs 148. A post 172 extends upwardly from the printed circuit board 170. A screw 174 that extends through lid horizontal surface 135 into post 172 holds the circuit board 170 to the lid.

Seen extending from circuit board 170 are two conductors 176. Conductors 176 provide an electrical connection between the cells 44 and the components on the circuit board 170. As discussed in more detail below, energization signals are continually applied to microcontroller 46 and temperature sensor 48 of battery 40 regardless of whether or not the battery is being charged, discharged, autoclaved, or simply in storage.

Also seen in FIG. 3 are the wire assemblies 177 that extend from the cell cluster to contacts 70. Also seen in the Figure but not otherwise described further are the button head fasteners 178 and lock washers 179 that hold the contacts 70 and 72 in position. Also seen is the O-ring 180 disposed around post 172.

FIG. 12 is a schematic of the electrical circuit components integral with the battery 40. A voltage regulator 182 is connected to the positive output terminal of the cell cluster 62. In one version of the invention voltage regulator produces a 3.3 VDC signal, the signal present at point 183. A capacitor 184, tied between the pin of the voltage converter 182 at which the 3.3 VDC signal is present and ground, filters the 3.3 VDC signal.

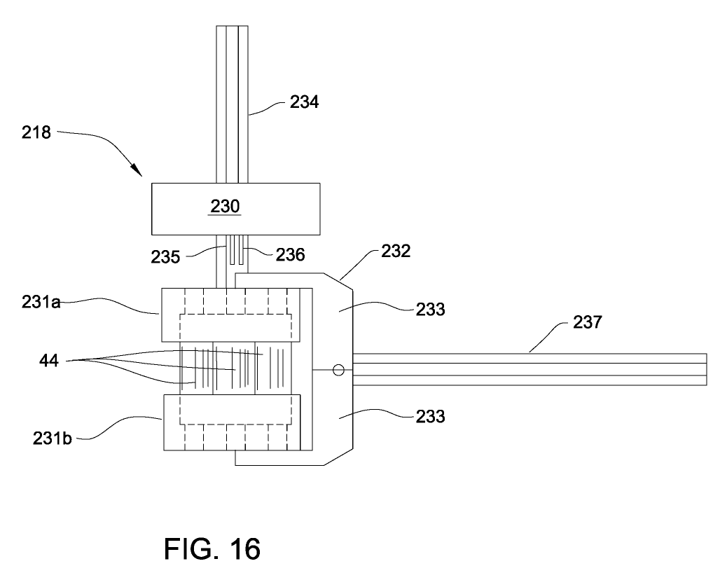

One of the components to which the 3.3 VDC signal is applied is the microcontroller 46. One suitable unit that can be used as microcontroller 46 is the P89LPC925 8 bit microcontroller manufactured by Philips Electronics N.V. of the Netherlands. Microcontroller 46 has a number of different sub-circuits, a number of which are now described by reference to FIG. 13. A central processing unit (CPU) 185 controls most of the operation of microcontroller 46 and the components connected to the microcontroller. A non volatile flash memory 187 stores instructions executed by the CPU 185. As discussed below, memory 187 also stores: the instructions used to regulate the charging of the battery; data describing the use history of the battery; and data describing the use history of the tool 522 to which the battery is attached.

A random access memory 188 functions as a temporary buffer for data read and generated by microcontroller 46. A CPU clock 189 supplies the clock signal used to regulate the operation of the CPU 185. While shown as single block for purposes of simplicity, it should be appreciated that CPU clock 189 includes an on-chip oscillator as well as sub-circuits that convert the output signal from the oscillator into a CPU clock signal. A real time clock 190 generates a clock signal at fixed intervals as discussed below.

The output signal from the temperature sensor is applied to both an analog comparator 191 and an analog to digital converter 192. In FIG. 13 the above sub-circuits are shown interconnected by a single bus 193. It should be appreciated that this is for simplicity. In practice, dedicated lines may connect certain of the sub circuits together. Likewise it should be understood microcontroller 46 may have other sub-circuits. These sub-circuits are not specifically relevant to this invention and so are not described in detailed.

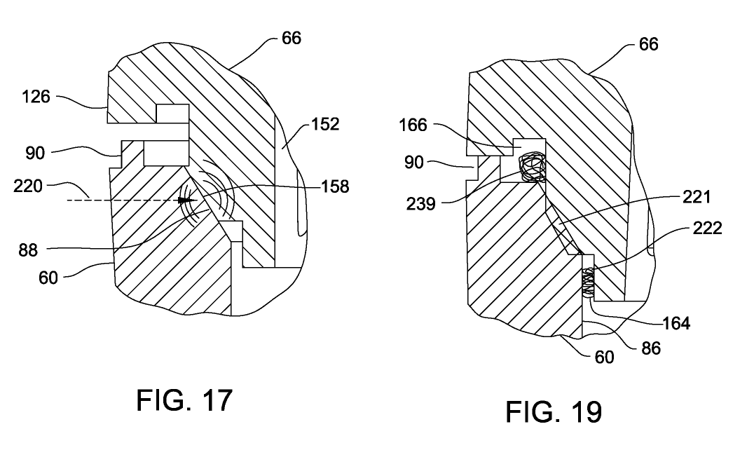

FIG. 14 illustrates types of data stored in the flash memory 187 in addition to the instructions executed by the microcontroller 46. These data include, in a field or file 194, data that identifies the battery. These data, in addition to serial number, lot number and manufacturer identification can include data such as an authorization code. This code is read by the tool 522 or charger 42 to which the battery is connected to determine if, respectively the battery can power the tool or be recharged by the charger. The battery identification data may include data indicating the useful life of the battery. Useful life data are understood to be one or more of the following data types: battery expiration data; number of chargings; and number of autoclavings. Other data in identification file 194 can indicate the nominal open circuit voltage of the signal produced by the battery, the current the battery can produce and the joules of available energy.

Charging instructions for the battery are stored in a file 195. These data can be the types of data described in the memories of the batteries disclosed in incorporated by reference U.S. Pat. Nos. 6,018,227, and 6,184,655. Flash memory 187 also contains data describing the charging and autoclave histories of the battery. In a field 196 data are stored indicating the number of times the battery was charged. A measured post-charge voltages file 197 contains data indicating the measured voltages-at-load of the battery after each charging. In some versions of the invention file 197 only contains these measurements for the last 1 to 10 chargings. In a file 198 data are stored indicating the highest battery temperature measured during its previous chargings. Again, file 198 may only contain data indicating the highest temperatures measured during the last 1 to 10 chargings of the battery.

A field 199 stores data indicating the total number of times the battery has been autoclaved. A cumulative autoclave time field 200, as its name implies, is used to store data indicating the total time the battery has been at temperatures at or above a threshold considered to be the autoclave temperature.

A field 201 contains data indicating the number of times the battery has been exposed to potentially excessive autoclavings. Data indicating the cumulative time the battery may have been potentially excessively autoclaved is stored in a field 202. A peak autoclave temperature field 203 contains data indicating the highest autoclave temperature to which has been exposed. A file 204 contains records of the time the battery has been in the autoclave for each of its autoclavings. In some versions of the invention, time in autoclave file 204 only contains data indicating the time the battery was in the autoclave for each of its last 5 to 100 autoclavings. A file 205 contains data indicating the peak temperatures of the battery that measured during its last 5 to 100 autoclavings. In most versions of the invention, memory 187 stores autoclave time and temperature data for the exact same number of autoclavings. Field 206 contains data indicating the period of the longest single time the battery was subjected to autoclaving.

There are also battery initial voltage and final voltage fields, fields 227 and 228, respectively. As there titles implies, fields 227 and 228, are respectively used to store data indicating the initial and final battery voltages when it is first connected to and finally removed the associated tool 522. Memory 187 also contains a tool history file 229. As discussed below, tool history file 229 stores data obtained from the tool 522 that battery 40 is employed to power.

Returning to FIG. 12, other circuit components internal to battery 40 are now described. Temperature sensor 48 is any suitable temperature sensing device capable of detecting whether or not battery 40 is exposed to autoclave temperatures. In the described versions of the invention, temperature sensor 48 is a thermistor. The 3.3 VDC is applied to one end of the temperature sensor. The opposed end of the temperature sensor 48 is tied to ground through a resistor 207. A capacitor 208 is tied across resistor 207. The voltage present at the junction of the temperature sensor 48 and resistor 207 is applied as the T_SENSE signal representative of detected temperature to the noninverting input of microcontroller comparator 191 (connection not specifically shown.)

A reference voltage, V_{TEMP}_{_{—}}_{REF}, is applied to the inverting input of comparator 191 (connection not specifically shown.) The reference voltage is the signal present at the junction of series connected resistors 209 and 210. The opposed end of resistor 209 receives a reference voltage from a source internal to microcontroller 46. The opposed end of resistor 210 is selectively tied to ground through a switch internal to the microcontroller 46 (switch not illustrated).

Microcontroller 48 is connected to battery contact 72 by a conductor 211. A pair of series-connected opposed diodes 212 extend between conductor 211 and ground.

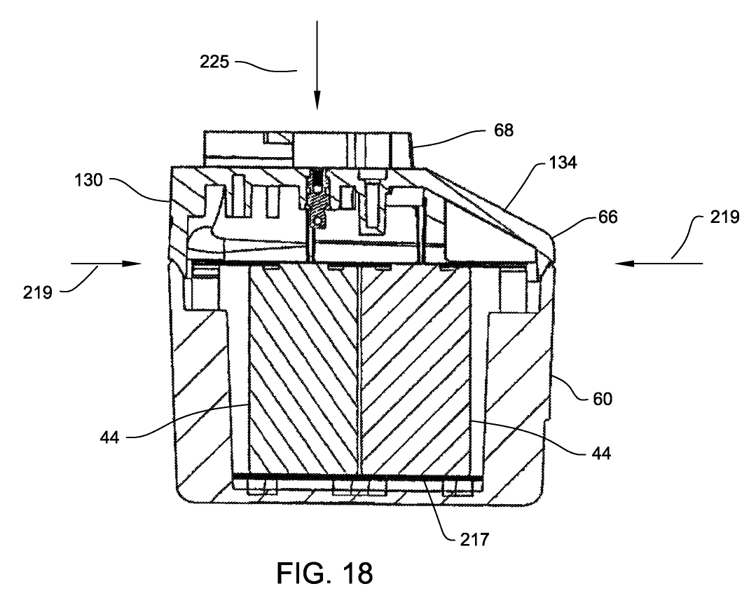

As part of the process of assembling battery 40, cell cluster 62 is assembled. Initially, binder assemblies 102 and 104 are fabricated as described above. Then, a first binder assembly 102 or 104 is placed in a fixture 213a or 213b, FIG. 15A illustrating fixture 213a, the fixture in which the top binder assembly 102 is seated. Each Fixture 213a and 213b includes a base plate 214 formed with a number of openings 215. A block 216 extends upwardly from the fixture base plate. Block 216 is shaped to define a recess 223 dimensioned to slip fit receive the binder assembly 102 or 104 and cells 44. The block 216 is formed to define the pattern of the rows 92, 94 and 96 in which the cells are to be placed. Illustrated fixture 213a is further shaped to define two opposed slots 224 that are contiguous with recess 223. Slots 224 receive the free end of the top binder assembly conductive straps 106 that function as electrical connections. Thus, fixture 213a has a supplemental block 216a spaced from block 216 so as to define slots 224 therebetween.

Fixture openings 215 are formed in the fixture base plate 214 to be concentric with the binder openings 112 and 114. When a cell is fitted in the fixture 213a or 213b it should be appreciated the cell is centered with binder openings 112 and 114 and the associated fixture opening 215.

The second binder assembly 104 or 102 is then placed in its associated fixture 213b or 213a, respectively. As seen in FIG. 15B, the second fixture with fitted binder assembly is then fitted over the fixture assembly in which the binder assembly 102 or 104 and cells 44 are already placed.

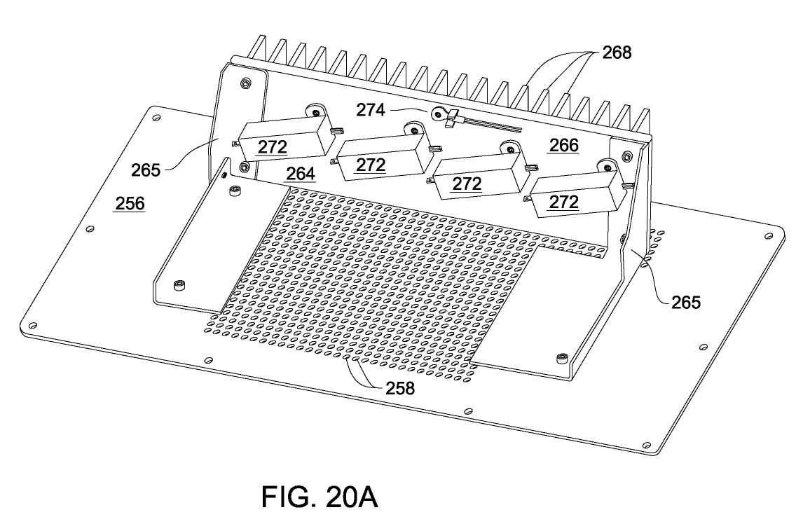

A robotic welding unit 218, shown diagrammatically in FIG. 16, welds the conductive straps 106 and fuse 118 to the cells 44. Specifically, robotic welding unit 218 has a base 237 to which an arm 232 is attached. Arm 232 includes two opposed fingers 233 that, when brought together, clamp cells 44 and fixtures 213a and 213b therebetweeen. A drive mechanism, (not illustrated,) moves arm 232 and the components held thereby both in the X plane (to the left and right in FIG. 16) and in the Y-plane (in and out of the plane of FIG. 16).

Robotic welding unit 218 also includes a welding head 230. Head 230 is attached to a track 234 so as to be able to move in Z-plane, (vertically in FIG. 16). Two opposed electrodes 235 and 236 are attached to and extending downwardly from head 230.

The welding process begins with the placement of the sandwiched-between-fixtures cells 44 and binders 102 and 104 between fingers 233 of arm 232. Arm 232 is moved so that a first one of the fixture openings 215 is disposed below electrodes 235 and 236. Welding head 230 is lowered so that the electrodes 235 and 236 pass through the fixture opening 215 and the aligned binder opening 114 to the surface of the exposed conductive strap 106 (or fuse 118). Current is flowed between the electrodes 235 and 236 to weld the strap 106 (or fuse 118) to the surface of the underlying cell 44. Once this weld process is complete, head 230 is raised. Arm 232 is slightly repositioned so that when head 230 is again lowered, electrode 235 and 260 can make a second weld joint between the same strap 106 (or fuse 118) and cell 44.

After the two weld joints for the first strap (or fuse) cell interface are completed, head 230 is again raised. Arm 232 is again positioned so each strap- (or fuse-) and-cell interface is similarly welded.

The final assembly of the battery 40 begins with the seating of a shock absorber 217 seen in FIG. 3, in the base of the housing 60. The shock absorber 217 is formed from a compressible material such as a silicon rubber. Shock absorber 217 subtends the area subtended by the cell cluster 62. In some versions of the invention, the shock absorber 217 is, in an earlier step bonded to the exposed face of the bottom binder assembly 104. Cell cluster 62 is placed in the housing. The connections are made between the cell cluster 62 and conductors 176 and wire assemblies 177.

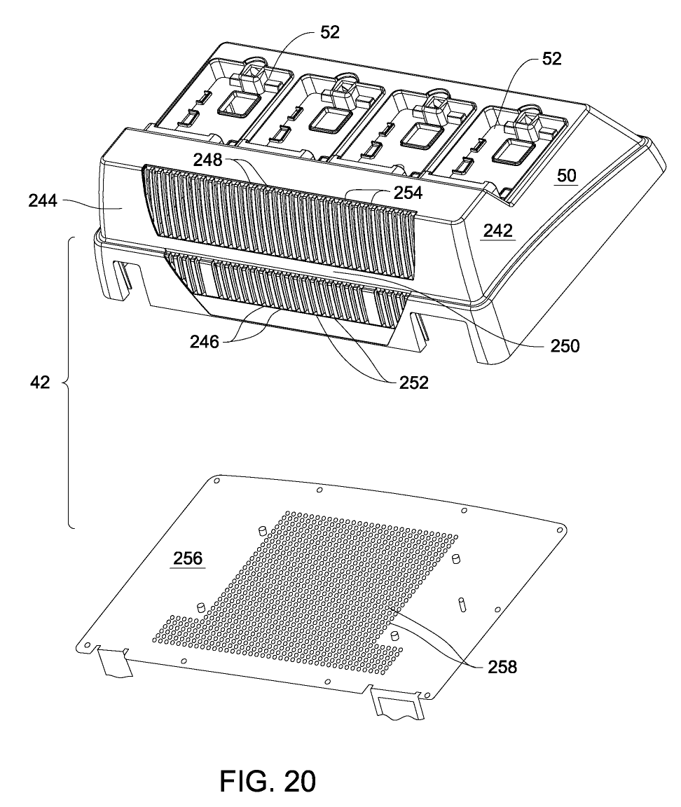

Lid 66 is then welded to the housing 60 to complete the assembly of the battery 40. In this process, the lid 66 is seated on the housing so that lid tapered surface 158 abuts housing tapered face 88. As seen in FIG. 17, owing to the dimensioning of housing 60 and lid 66, at this time, the lid is positioned so that the bottom horizontal surface of the lid base 126 is spaced above the housing reveal 90.

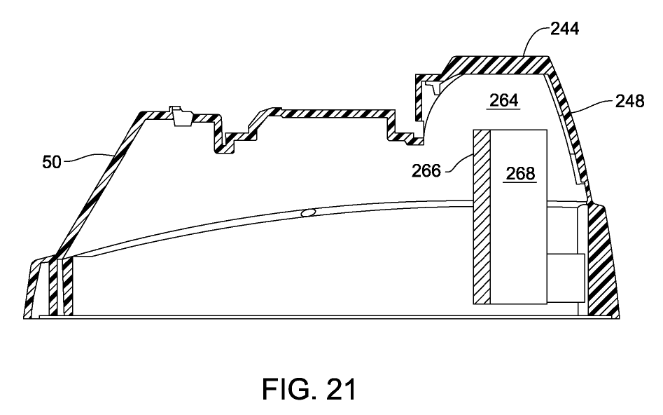

The welding process is accomplished by applying a downward force on the lid 66 so that the lid bears against the housing 60. In FIG. 18, this is represented diagrammatically by arrow 225. More particularly, owing to the angled profile of housing tapered surface 88 and lid tapered surface 158, these surfaces 88 and 158 abut. Simultaneously with application of the downward force, coherent (laser) light at 980 nanometers is simultaneously applied to the lateral section of the housing that subtends the interface between housing tapered face 88 and lid tapered surface 158. As represented by plural arrows 219, this photonic energy is applied simultaneously around the whole of the perimeter of the outer housing. A suitable system capable of performing this welding is available from Branson Ultrasonics of Danbury, Conn.

Owing to the transmissivity of the material forming the housing 60 to this wavelength of photonic energy, the energy passes substantially through the housing lip 84 as represented by phantom arrow 220 of FIG. 17. This energy is absorbed by the material forming lid lip 152. The material forming lid lip 152 thus heats to its melting point. This includes the material forming lid tapered surface 158. Owing to the downward force imposed on the lid 66, the lid therefore settles downwardly into the open space of the housing 60. The settling of lid 66 stops by the abutment of the bottom surface of lid base 126 against housing reveal 90.

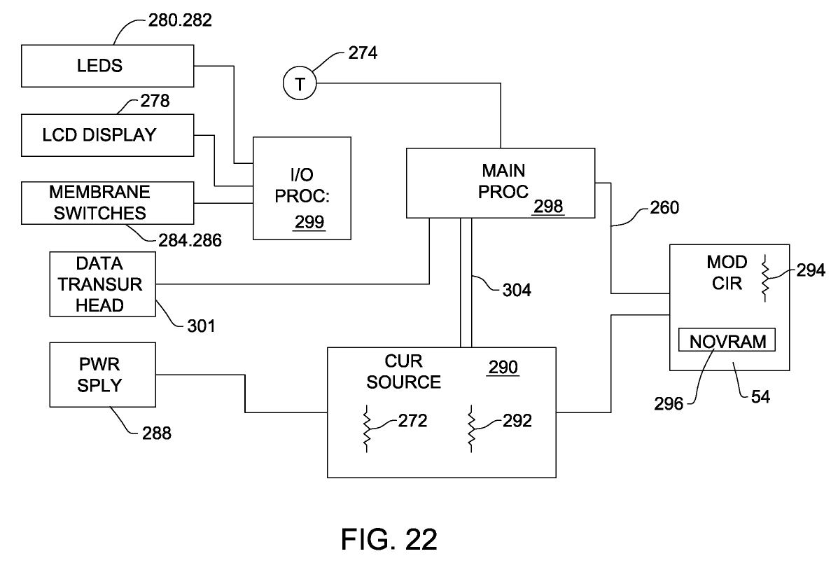

Moreover, thermal energy is transferred from the lid tapered surface 158 to the adjacent abutting housing tapered surface 88. As represented to FIG. 19, this causes the material forming the housing tapered face 88 to likewise melt. Collectively, the material forming the opposed housing tapered face 88 and lid tapered surface 158 form a hermetic weld joint 221 around and along the interface of the battery housing 60 and lid 66.

It should be appreciated that, as part of the above process, a small amount of the material forming the housing tapered face 88 and lid tapered surface 158 spread away from these two surfaces. Some of this material, flash material 239 in FIG. 19, flows into the space immediately inward of housing reveal 90 and the contiguous lid notch 166. Other of this material, flash material 222, flows into the space between housing vertical surface 86 and lid lip outer vertical surface 164.

### III. Charger

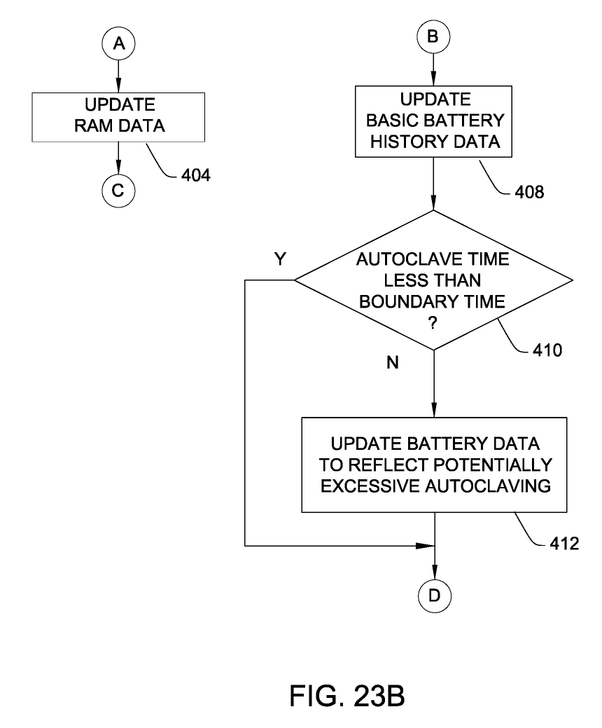

The basic structure of the battery charger 42 is now explained by reference to FIGS. 20, 20A and 21. Pockets 52 are formed in a front flat portion of the charger housing 50, (flat portion not identified). The charger housing 50 is further formed to have a back section 242 that is raised relative to the section in which pockets 52 are formed. A rear wall 244 forms the rear end of section 242 and thus, the rear end of the charger housing 50. Housing rear wall 244 is formed with a set of lower and upper ribs 246 and 248, respectively. Both ribs 246 and 248 extend vertically. A web 250, part of housing rear wall 244, separates ribs 246 and 248 from each other. Ribs 246 are spaced apart from each other to define vertical vents 252 therebetween. Ribs 248 are spaced apart from each other to define vertical vents 254 therebetween.

Battery charger 42 also has a metallic, plate shaped base 256. In one version of the invention, the base 256 is formed from spring steel. Base 256 is disposed in the open end of housing 50. The base 256 is shaped to have numerous openings 258 that extend therethrough. Base 256 is the structural component internal to the charger to which the majority of other charger components are attached. Not seen are the structural components and fasteners that hold housing 50 and base 256 together.

One component attached to base 256 is a heat sink 264. In some versions of the invention, heat sink 264 is formed from aluminum or other material with good thermal conductivity characteristics. The heat sink 264 is shaped to have a planar base 266. A number of fins 268 extend perpendicularly outwardly from the base 266. Fins 268 extend laterally across the base 266.

The heat sink 264 is mounted to base 256 by brackets 265. More particularly, the heat sink 264 is mounted to the base 256 so that the heat sink is disposed within the space internal to housing back section 242. More particularly the heat sink 264 is positioned so that there is free space between the outer edges of the fins 268 and housing vents 252 and 254.

A set of discharge resistors 272 are mounted to the face of the heat sink base 266 opposite fins 268. As discussed below, during certain processes for charging or evaluating a battery 42, it is necessary to first fully discharge the stored energy in the battery. This process step is executed by connecting the battery to a discharge resistor 272. In the illustrated version of the invention, each discharge resistor 272 is associated with a separate one of the charger pockets 52. During the discharging of a battery 40, each battery is tied to the specific discharge resistor 272 associated with the pocket in which the module 54 to which the battery is coupled is seated.

Each discharge resistor 272 generally has a resistance of 15 Ohms or less. In still other versions of the invention, each discharge resistor 272 has resistance of 10 Ohms or less. Each discharge resistor 272 is often encased in its own heat sink, (not illustrated). This resistor heat sink is the resistor component that physically abuts the heat sink base 266.

Also attached to the heat sink base 266 is a temperature sensor 274. It will be observed there is no fan or other device internal to or otherwise integral with the charger 42 for moving air through the housing 50 or across the heat sink 264.

From FIG. 1 it is seen that each I/O unit 58 includes an LCD display 278 and two LEDs 280 and 282. Each I/O unit 58 of charger 40 of this invention further includes two membrane switches 284 and 286.

FIG. 22 is a block diagram of the electric circuit assemblies internal to charger 42. A power supply 288 converts the line current into signals that can be used to energize the other components internal to the charger 42. Power supply 288 also produces a signal that is applied, through a module 54 to the battery 40 to charge cells 44.

The charging current is applied to the battery by a current source 290. In actuality, charger 42 has plural current sources 290; one to apply current to a battery through each module 54. This allows different charging signals to be applied to simultaneously to separate attached batteries. For simplicity, only a single current source 290 is illustrated. Integral to each current source 290 is a resistor 292. When the battery 40 is seated in module 54, resistor 292 establishes a connection between the battery positive terminal and ground. Each discharge resistor 272 is associated with a separate one of the current sources. Thus, in FIG. 22, the discharge resistor 272 is shown internal to the current source 290. Each discharge resistor 272 has one end selectively connected to ground. The opposed end of resistor 272 is selectively tied to the battery positive terminal by a switch, typically a FET (switch not shown).

Module 54, one shown as a block element in FIG. 18, also includes a resistor 294. Resistor 294 is selectively connected across the terminals to which battery contacts 70 are connected. A switch, typically a FET (not illustrated) is used to make this connection. Resistor 294 is thus used to measure the voltage at load of the battery 40.

The module 54 also contains a NOVRAM 296. NOVRAM 296 contains charging sequence and charging parameter data used to regulate the charging of the battery 40 charged through the module. A main processor 298, also internal to charger 42, controls the charging of the battery 40. Main processor 298 further determines, if it is necessary to perform a state of health evaluation of a battery, performs the evaluation and, based on the data generated in the evaluation, generates an indication of the state of health of the battery. Main processor 298 also generates the read/write instructions to obtain data from and load data into the memory integral with battery microcontroller 46 and module NOVRAM 296. In one version of the invention, the AT91SAM7X256/128 available from Atmel of San Jose, Calif. functions as the main processor 298.

More specifically, the main processor 298 is connected to the current source 290 over a plurality of conductors collectively represented as bus 304. Main processor 298 outputs a variable CURRENT_CONTROL signal to the current source 290. In response to the CURRENT_CONTROL signal, current source 290 outputs a charging current, at a select current, through module 54 to the battery cells 44. The voltage across resistor 292 is output over bus 304 to the main processor 298 as a MEASURED_VOLTAGE signal. This MEASURED_VOLTAGE signal is representative of the voltage across the battery 40. Also output from the main processor 298 through bus 304 is the signal to the switch that selectively ties resistor 272 to the battery 40. This connection causes the charge stored in the battery 40 to be discharge by the resistor 272.

Main processor 298 is connected to the module 54 by a plurality of conductors represented as a single-wire bus 260. Main processor 298 selectively generates the control signal that connects resistor 294 across the positive and negative terminals of the battery 40. When resistor 294 is so connected, the resistor 294 is connected to resistor 292. The MEASURED_VOLTAGE signal from the current source 290 thus becomes a measure of the voltage at load of the battery 40.

Bus 260 also functions as the link through which the contents of the module NOVRAM 296 are written to main processor 298. Data are also read from and written to the battery microcontroller 46 over bus 260.

The output signal produced by temperature sensor 274 is applied to the main processor 298.

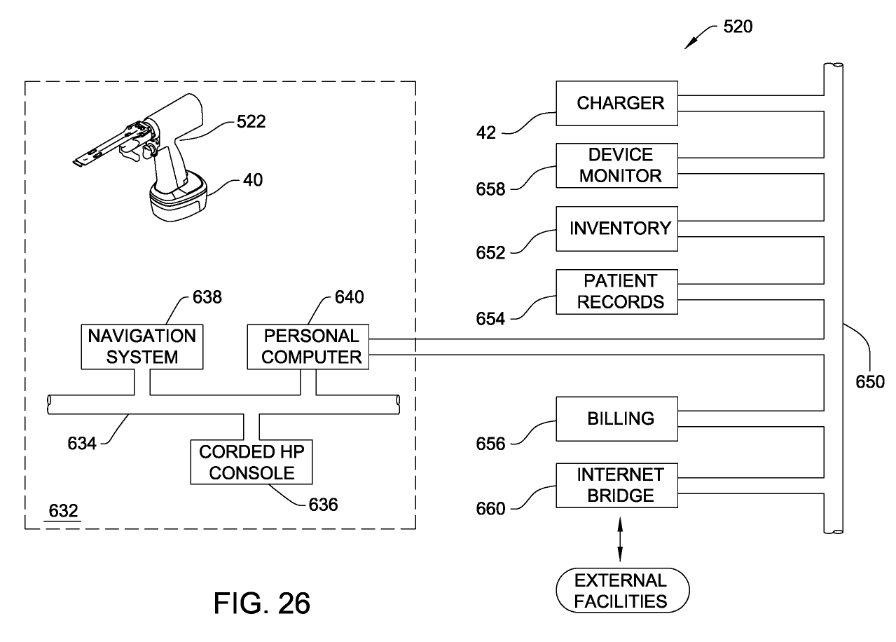

Main processor 298 is also connected to a data transceiver head 301. Transceiver head 301 is the interface internal to the charger connected to bus 650 (FIG. 26). As described below, this allows data regarding the battery 40 and tool 522 energized by the battery to be collected and forwarded to persons responsible for ensuring their availability.

A more detailed description of the components internal to module 54 and current source 290 as well as the processes by which a battery may be charged is disclosed in the incorporated by reference U.S. Pat. No. 6,018,227. Additional description of the processes involved in charging plural batteries and alternative charge assemblies are found in the Applicants' Assignee's U.S. Pat. No. 6,184,655, Battery Charging System With Internal Power Manager, issued 6 Feb. 2001, the contents of which is incorporated herein by reference.

Battery charger 42 also contains an I/O processor 299. The I/O processor 299, based on signals output from the main processor 298, generates the signals that cause LCD display 278 to generate the appropriate image. The I/O processor 60 also regulates actuation of the LEDs 280 and 282. Membrane switches 284 and 286 are also connected to the I/O processor 299. Based on the signal generated as a consequence of the opening and closing of switches 284 and 286, the I/O processor 299 generates the appropriate commands to the main processor 298.

### IV. Operation

A. Battery

Battery microcontroller 46 operates in three different modes. This is to minimize the load the components internal to the battery 40 place on cells 44. In a normal mode, all subcircuits internal to the microcontroller 46 are energized. In one version of the invention, when microcontroller 46 is in this state, it draws approximately 6 mA. Microcontroller 46 also has a power down, clock on state. When the microcontroller 46 is in this state, CPU 185, analog comparator 191 and the analog to digital circuit 192 are deactivated. Both the CPU clock 189 and the real time clock 190 are on when microcontroller 46 is in the power down, clock on state. When microcontroller 46 is in the power down, clock on state, the microcontroller draws approximately 3 mA.

A power down, clock off state is the lowest power consuming state in which microcontroller 46 operates. In this state, the CPU 185, the CPU clock 189, the real time clock 190 and the analog to digital circuit 192 are deactivated. When microcontroller 46 is in this state, the analog comparator 191 is activated. When microcontroller 46 is in the power down, clock off state, it draws approximately 120 to 150 μA.

It should further be appreciated that during the states in which the analog comparator 191 is on, switches internal to microcontroller 46 are set so there is current flow through resistors 209 and 210 to ground. This results in a V_{TEMP}_{_{—}}_{REF} signal appearing at the inverting input of the comparator. When the analog comparator 191 is turned off, when battery microcontroller 46 is in the power down, clock on state, the microcontroller switches are set so both resistors 209 and 210 are tied high. This eliminates current draw of these resistors.

The operation of microcontroller 46 is now explained by reference to the flow chart of FIGS. 23A and 23B. For the majority of the time, battery microcontroller 46 is in the power down, clock off state. In FIG. 21A this is represented by step 390, the microcontroller entering the power down, clock off state.

When microcontroller 46 is in this state, analog comparator 191 continually compares the V_{TEMP} to V_{TEMP}_{_{—}}_{REF}, step 392. As long as this comparison indicates that signal from temperature sensor 48 indicates that the battery is not being autoclaved, microcontroller 46 remains in the power down, clock off state.

It should be appreciated that the reference signal V_{TEMP}_{_{—}}_{REF} may not be a signal that corresponds to the actual temperature inside the autoclave. Instead to compensate for the thermal insulation of the battery housing 60 and lid 66, the V_{TEMP}_{_{—}}_{REF} may be at a level that corresponds to a temperature less than that of the actual autoclave temperature. In some versions of the invention, the V_{TEMP}_{_{—}}_{REF} signal is set to level to be representative of an autoclave temperature, generally this is an ambient temperature, of at least 100° C. Often, this is an ambient temperature of between 100 and 150° C. In alternative versions of the invention, it may be desirable to set the V_{TEMP}_{_{—}}_{REF} signal so that the battery is considered in a harsh environment when in environment when the ambient temperature is at least 70° C. The actual level of the V_{TEMP}_{_{—}}_{REF} signal may be determined by thermal modeling and/or empirical analysis.

If, in step 392, the comparison indicates that V_{TEMP} is above V_{TEMP}_{_{—}}_{REF}, microcontroller 46 interprets V_{TEMP} signal as indicating that the battery is being subjected to autoclaving. In response to this event, microcontroller 46, in step 394, enters the power down, clock on mode.

As a result of the microcontroller 46 entering the power down, clock on mode, the real time clock 190 counts down a 30 second time period, step 396. At the conclusion of this count, the microcontroller 46 transitions to the normal mode, step 398. Once in the normal mode, in a step 402, using comparator 191 again compares V_{TEMP} to V_{TEMP}_{_{—}}_{REF}.

If the comparison of step 402 indicates that the battery is still being autoclaved, CPU 185 performs a data update step 404. In step 404, data stored in RAM 188 are updated. These data include a field that indicates the total time the battery has been at autoclave temperature. In some versions of this invention, the data in this field is simply incremented by a unit count (one unit=30 sec.). Also data in a RAM field that indicates the highest temperature of the current autoclave cycle may be updated. In this part of step 402, a digital signal representative of the V_{TEMP} from the analog digital converter 192 are compared to the stored temperature level in the RAM 188. If the data from converter 192 is representative of a higher temperature than the stored measurement, these data are overwritten into the RAM field.

Once step 404 is executed, microcontroller 46 reenters the power down, clock on mode. Thus steps 394, 396 and 402 are reexecuted.

Upon completion of the autoclave process, battery temperature will drop to below the autoclave temperature. This event will be indicated by a different result in the comparison of step 402. Battery microcontroller 46 then updates the data stored in memory 187. This process includes an updating of the basic history data stored in memory 187, step 408. As part of step 408, then count of the number of times the battery has been autoclaved, the data in field 199 is incremented by one. Based on the data in the RAM 188 indicating the total time the battery was autoclaved, the data in the cumulative autoclave time field 200 is likewise revised. Also in step 408, the data in field 204 is updated to indicate the time the battery was, in this last autoclaving, autoclaved.

In step 408, the data in memory 187 are updated based on the RAM data indicating the total time the battery was, in this autoclaving autoclaved. Specifically, data indicating the total time the battery was autoclaved in this cycle are written into field 205. The data in field 206 indicating the peak single autoclave time is, if necessary, likewise rewritten. In some versions of the invention these data are first written into the RAM.

In a step 410 microcontroller CPU 185 determines if the battery was subjected to a potentially excessive autoclaving. This step is performed by comparing from RAM 188 the time the battery was autoclaved to a boundary time. This boundary time is the limit of the acceptable time for which the battery can be autoclaved and there will not be any potential of damage to its internal components. In some versions of the invention, this boundary time is between 3 and 60 minutes. In still more preferred versions of the invention, this boundary time is between 5 and 30 minutes.

If the battery was not subjected to a potentially excessive autoclaving, microcontroller returns to the power down, clock off mode. Step 390 is reexecuted.

However, if the comparison of step 410 indicates that the battery may have been subjected to a potentially excessive autoclaving, there are further revisions to the data in a step 412. In step 412 the data in field 201 indicating the number of potentially excessive autoclaving to which the battery was subjected is incremented. In some versions of this invention these data are first written into the RAM 188. Then, in a single write-to-flash step, (not illustrated,) all the data written to the RAM 188 in steps 408 and 412 are written to the flash memory 187.

Also in step 412, the cumulative time to which the battery has been exposed to potentially excessive autoclaving is updated. This time count is first adjusted by subtracting from the total time of the battery was autoclaved the boundary time. Thus, if the battery was autoclaved for 12 minutes and the boundary time was 10 minutes, by subtraction the CPU 185 determines that for this autoclave cycle the battery was subjected to 2 minutes of potentially excessive autoclaving. This is the value added to the cumulative data stored in field 202. Step 390 is then executed to return battery microcontroller 46 to the power down, clock off state.

B. Charger

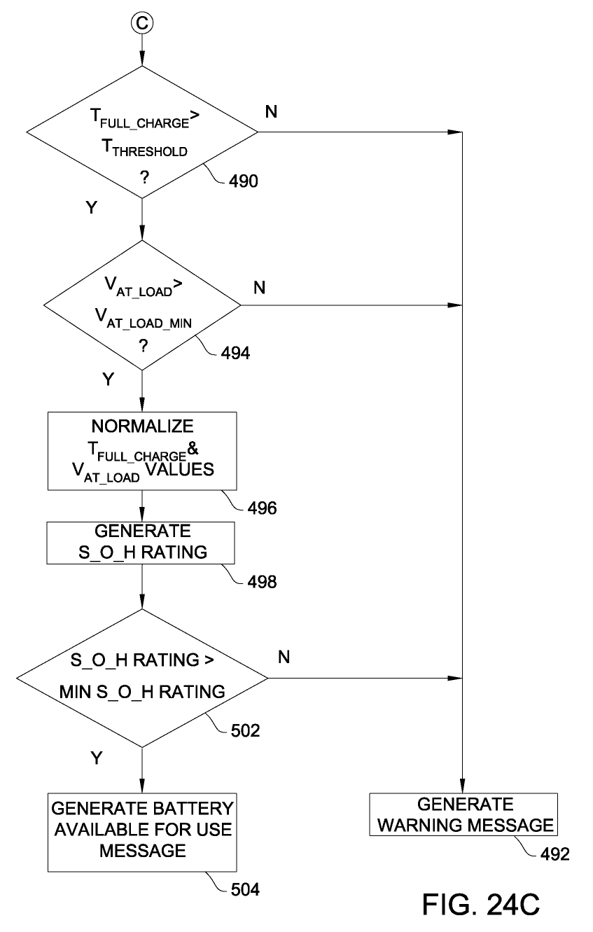

The process by which the charger 42 charges the battery is now 40 is now described by reference to the flow charts of FIGS. 24A, 24B and 24C. While not illustrated, it should be understood that the depicted process assumes the module 54 is seated in a charger pocket 52. Upon the seating of each module 54 in a pocket 52, the data in the module NOVRAM 296 are read to the charger main processor 298, (step not shown). In a step 452, main processor 298 continually tests to determine if a battery 42 is seated in a module 54. This test is performed by monitoring the level of the current source MEASURED_VOLTAGE signal. Specifically, if a battery is not seated in a module 54, the MEASURED_VOLTAGE signal is the open circuit voltage of the charging signal output by the current source. In some embodiments of the invention, this voltage is 20 VDC. As long as the MEASURED_VOLTAGE signal remains at the open circuit voltage level, main processor 298 continually reexecutes step 452.

The seating of a battery 40 in the module 54 causes the MEASURED_VOLTAGE signal to drop. In response to the drop in this signal level, (the seating of the battery in the module,) in a step 453 main processor 298 causes battery microcontroller 46 to transition from the power down, clock off mode to the normal mode. In one version of this invention, this transition is effected by tying battery contact 70 to ground for a given time period. This pulls the one-wire communication line connected to microcontroller 46 to ground. An interrupt circuit internal to battery microcontroller 46 (circuit not illustrated) continually monitors this communication line. The interrupt circuit interprets the extended low state signal on the communication line as indication that it should transition the microcontroller 46 from the power on clock off state to the normal state.

Once the battery microcontroller 46 is in the normal mode, main processor 298 generates an instruction through the module 54 to cause the battery microcontroller 46 to write out to the main processor 298 the contents of the associated memory 187. These data are written out to the main processor 298. The data written to the charger processor 298 include the charging sequence instructions and the data describing the use and autoclave history of the battery. Collectively, this read request and data write out are shown as step 456.

Main processor 298 then determines if the data retrieved from memory 187 indicates the battery should be subjected to a full state of health (S_O_H) evaluation. One test made to determine if the battery 40 should be so evaluated is, in step 458, the determination based on the data retrieved from memory file 204. The last entry in file 204 indicates the total time the battery was autoclaved in the last autoclaving. Main processor 298, in step 458 compares this value to the boundary time. If the last autoclaving was for a time more than the boundary time, the main processor 298 considers the battery to be in a state in which it is appropriate to perform a state of health evaluation.

As represented by step 460 other data read from the battery memory 187 are also tested to determine if a state of health evaluation is required. For example, in step 460 the data in the fields 196 and 199 are read to determine if, respectively, the battery has been subjected to more than P number of rechargings or Q number of autoclavings. Also in step 460 the data in field 202 are read to determine if, since manufacture, the battery has been subject to R amount of total time of potential excessive autoclave exposure. It should be appreciated that, in step 460, processor 298 determines it is necessary to perform a complete state of health evaluation if the battery has been subjected to a multiple of P rechargings, Q autoclavings or R total time of potentially excessive autoclave exposure.

Also once the charger processor 298 detects the battery is placed in the module socket 56, the processor may cause a message to be presented on the complementary display 278 asking if a state of health evaluation is wanted, (step not shown). The person responsible for charging the battery 40 indicates if the evaluation is required by depressing an appropriate one of the membrane switches 284 or 286, step 462.

If a state of health evaluation is not required, the charger executes a standard charging sequence for the battery, step 464. In step 464, based on the sequence instructions received from the battery microcontroller memory 187 or module NOVRAM 296, charger main processor 298 causes the connected current source 290 to apply the appropriate sequence of charging currents to the battery cells 64. It should be appreciated that the charging currents are also based on the MEASURED_VOLTAGE signals obtained from the current source 290.

Once the charging process is complete, charger 42 performs a voltage at load test on the battery, step 466. Typically, the voltage at load test is performed by measuring the voltage at load across the battery 40. Charger main processor 298 performs this evaluation by asserting the appropriate gate signal to FET integral with the module to which resistor 294 is attached (FET not illustrated). This results in the connecting of the module resistor 294 across the positive and negative terminals of the battery. As a result of resistor 294 being so connected to the battery, the MEASURED_VOLTAGE signal from the current source 290 becomes a measure of the voltage-at-load of the battery. Execution of this single test of battery state can be considered the performance of a partial state-of-health evaluation of the battery 42.

In a step 468, main processor 298, through I/O processor 299, causes an image to be presented on display 278 indicating the voltage at load of the battery. This data is sometimes referred to as an indication of the basic state of health of the battery. If the battery voltage at load (basic state of health) is at or above an acceptable level, main processor 298, again through the I/O processor 299, causes an appropriate one of the LEDs 280 or 282 to illuminate to indicate the battery is available for use, also part of step 468.

In a step 470, main processor 298 writes into battery memory 187 data regarding the charging. Specifically, in step 470 the count of the number of chargings stored in memory field 196 is incremented. Also data are added to file 197 to indicate the measured voltage-at-load of the battery after charging.

Eventually, the battery 40 is removed from the charger 42, step 471. As a consequence of this step, there is no communication over the one-wire line internal to the battery 40. The signal on this line transitions to a continuous high level state. As discussed above with respect to step 453, the signal level on this communications line is monitored by an interrupt circuit. The interrupt circuit interrupts the signal level of the communications line being high for an extended period of time as an indication that step 471 was executed. Therefore, in step 472, the interrupt circuit transitions the battery microcontroller from the normal state back to the power down, clock off state. Charger 42 returns to step 452.

While not shown, it should be understood that after the charging process is completed, main processor 298 also causes one of the LEDs to be appropriately actuated to indicate that the battery is available for use.

As represented by step 478, a battery full state of health evaluation starts with the complete discharging of the battery. Step 478 is executed by the main processor 298 asserting the appropriate gate signal to tie the battery positive terminal to resistor 272. Periodically, the voltage across the battery is measured, step 480. This step is executed until it is determined the battery is fully discharged.

Once the battery 40 is fully discharged, charger 42 proceeds to charge the battery, step 484. Step 484 is essentially identical to step 464. As part of this evaluation, main processor 298, in step 484, also monitors the overall length of time it takes for the cells 64 internal to battery to fully charge. As is known in the art, main processor typically determines the cells are full charged by determining when change in voltage over a period time falls to a value less than 0, (negative slope.) Thus, in step 486 during the primary or main state charging of the battery 40, main processor 298 monitors both the ΔV_{BATTERY}/ΔTime and the time from the start of the main state charging it takes for this slope to go negative. This time is T_{FULL}_{_{—}}_{CHARGE}.

Once the main state charging of the battery is complete, charger 42 performs a voltage at load test, step 488. Step 488 is essentially identical to the voltage at load test of step 466.

Based on the data obtain in steps 486 and 488, main processor 298 determines if the health of the battery is such that it can supply the amount of power needed to actuate a powered surgical tool. In a step 490, main processor 298 makes this determination by determining if the overall time it took the battery to fully charge, T_{FULL}_{_{—}}_{CHARGE}, is at or above a threshold time, T_{THRESHOLD}. The basis for this evaluation is that the T_{FULL}_{_{—}}_{CHARGE} time is directly proportional to the quantity of charge being stored in the battery. Therefore, if T_{FULL}_{_{—}}_{CHARGE}>T_{THRESHOLD}, this is an indication that the quantity of charge in the battery is above that needed to energize a surgical tool for the total time such power is required. Thus, when the above determination tests true, main processor 298 recognizes the battery as being in state in which it most likely can power the surgical instrument as required.

If the determination of step 490 tests false, main processor 298 considers the battery to be in the opposite state. In this event, main processor 298 causes the I/O processor 299 to generate the appropriate fault state message, step 492, regarding the battery 40 on the display 278. This provides notice the battery may not function appropriately.

As part of the state of health evaluation, main processor 298 determines whether or not the voltage at load is above a minimum voltage value, step 494. If the battery voltage at load is not above this minimum value, the battery is considered to have an internal resistance so high that it cannot appropriately energize the tool to which it is attached. Therefore, if in a step 494 the determination tests false, step 492 is executed.

As part of the state of health evaluation, main processor 298 further determines whether or not the battery can deliver sufficient charge based on both T_{FULL}_{_{—}}_{CHARGE} and the measured voltage at load. Specifically, both T_{FULL}_{_{—}}_{CHARGE} and measured voltage at load values are normalized, step 496. In some version of the invention, each of these values is normalized by quantifying them to a range for example, between 0.0 and 1.0.

Then, in a step 498 the normalized T_{FULLCHARGE} and V_{ATLOAD} values are used as input variables into an equation. This equation may be a simple summation,
S_{—}H_{—}R=T_{FULLCHARGE}+V_{ATLOAD} (1)
Here S_H_R is state of health result. Alternatively, the normalized values are multiplied by coefficients
S_{—}H_{—}R=A(T_{FULLCHARGE})+B(V_{ATLOAD}) (1a)
Here, A and B are constants. In some versions of the invention, the variables are multiplied together:
S_{—}H_{—}R=C(T_{FULLCHARGE})(V_{ATLOAD})+D (1b)
Here, C and D are constants.

Once S_H_R is calculated, in step 502, it is compared to a cutoff value, S_H_R_{CUTOFF}. If S_H_R is equal to or greater than S_H_R_{CUTOFF}, the charger main processor 298 recognizes the battery as being in a state in which it will deliver an appropriate charge to a surgical tool. Therefore, a step 504 is executed to cause the appropriate image to be presented on the display 282 and LED activation to indicate the battery is available for use. Also in step 504 the charger presents on display 278 an indication of the above calculated S_H_R result. These data are referred to as an indication of the calibrated state of health of the battery. If, in step 502 it is determined that the calculated S_H_R value is less than S_H_R_{CUTOFF}, step 492 is executed.

After either step 492 or 504 is executed, step 470 is executed to complete the charging process. (Not shown is the loop back to step 470.)

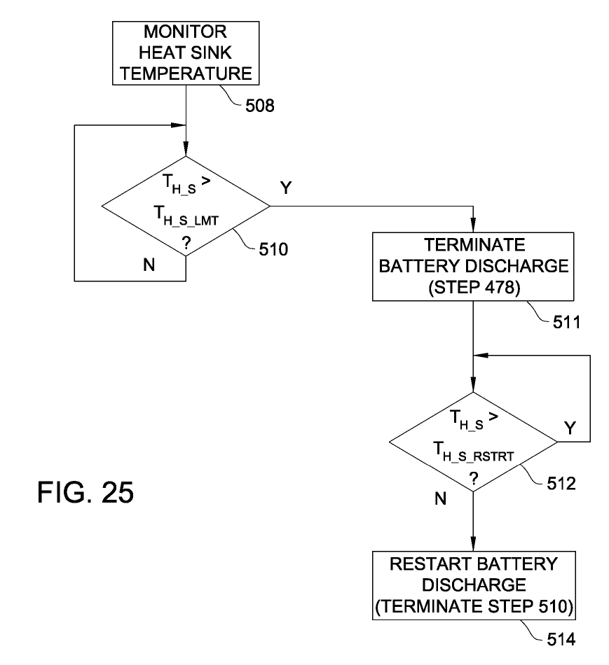

Charger 42 of this invention is further configured so that when actuated, temperature sensor 274 provides a signal to main processor 298 representative temperature of the heat sink 264. As represented by step 508 of FIG. 25, main processor 298 monitors the heat sink temperature, T_{H}_{_{—}}_{S}. As represented by step 510, the main processor compares the heat sink temperature to a limit temperature, T_{H}_{_{—}}_{S}_{_{—}}_{LMT}.

When charger 42 of this invention is required as part of a charging process or a state of health evaluation to discharge a battery 40, the battery charge is discharged through one of the resistors 272. The heat generated by this resistor is conductively transferred to heat sink 272. Most of the time air flow into the charger housing through base openings 258 and housing vents 252 has sufficient thermal capacity to sink the heat radiated by heat sink 272. This warmed air is discharged through housing vents 254. During such time periods the heat sink temperatures stays below the heat sink limit temperature.

However, there may be times the air flow past the heat sink 264 cannot sink all the heat sourced by the heat sink 264. This may occur if, due to unusual circumstances, the charger simultaneously discharges large amounts of current from plural batteries. If this event occurs, the measured rises heat sink temperature rises. If the heat sink temperature rises above the limit temperature, T_{H}_{_{—}}_{S}_{_{—}}_{LMT}, there is a possibility that further temperature rise will result in the charger housing 52 being heated to a temperature that makes it unpleasant, or worse, to touch the charger 42. The limit temperature, T_{H}_{_{—}}_{S}_{_{—}}_{LMT}, it should be appreciated, is often determined by empirical analysis.

Therefore, if the comparison of step 510 indicates the heat sink temperature is above the limit temperature, main processor 298 executes a battery discharge interrupt sequence represented by step 510. In this sequence, the charger interrupts the discharging of one or more attached batteries 40. Thus, in step 510, the discharge step 478 to which one or more of the batteries is presently being subjected may be interrupted. Similarly, if one of the batteries is being discharged as part of the normally charging sequence for that battery, that discharge step may likewise be interrupted.

Step 510 is executed until, as a result of a subsequent measurement of heat sink temperature, (step not shown) it is determined heat sink temperature has dropped below a restart temperature, T_{H}_{_{—}}_{S}_{_{—}}_{RSTRT}, step 512. Once the heat sink temperature is fallen to this level, additional thermal energy sourced by the discharged resistors 272 can be output without the likelihood of such heat placing the charger in an undesirable state. Therefore, once the heat sink temperature so drops, step 510 is terminated.

Battery 40 of this invention provides an indication if its cells may have been damaged. If the battery 40 may be in this state, charger 42 conducts a state of health evaluation on the battery. One immediate advantage of this invention is that, if the battery cells may have been damaged, a state of health evaluation is performed. This substantially reduces the possibility that someone will attempt to use a damaged battery to energize a surgical tool.

During the charging or discharging of the battery 40, the temperature of cells 44 inevitably rises. In this invention, each cell has some surface area that is spaced free of the adjacent cells. This minimizes the uneven heat dissipation and consequential uneven temperature rises of the cells. The reduction of this temperature imbalance results in a like lessening of the extent to which the individual cells 44 can become electrically imbalanced. Reducing the electrical imbalance of the cells reduces the extent to which the cells being so imbalanced can adversely affect either the utility or useful lifetime of the battery.

Battery 40 of this invention is also designed so that the narrow section 119 of fuse 118 is spaced from the adjacent binders 108 and 110. Section 119 is the section of the fuse 118 that vaporizes upon the flow of more than the selected amount of current flow through the fuse. Since fuse section 119 is not in physical contact with another section of the battery, no other section of the battery, such as the binders, serve as sinks for the heat generated by the current flow. Thus when the defined current flows through the fuse 118 the thermal energy generates in the vicinity of fuse section 119 stays in the section. This thermal energy therefore causes the fuse section 119 to rise to the level at which vaporization occurs. Thus, this design feature of the battery of this invention increases the likelihood that, when more than the defined current flows through the fuse, the fuse will open.

The charger 42 is further configured that it does not always perform the state of health evaluation, which can be time consuming to perform. Instead, the charger of this invention only performs this evaluation when the environmental history stored in the battery indicates it is desirable to perform the evaluation. By minimizing the number of times the charger performs state of health evaluations, the time it takes the charger to charge batteries is likewise held to a reasonable time period.

Still another feature of charger 42 is that the charger discharges batteries as part of a charging sequence or state of health evaluation yet it does not include a fan or other powered ventilation unit to exhaust air heated as a consequence of this discharging. The absence of fan in this charger reduces the noise generated by the charger when it is active. In the event there is an excessive generation of heat, further battery discharging is limited until the heat is dissipated.

Also battery 40 invention stores data regarding the environment to which the battery has been exposed. This information can be used to help evaluate why a battery underperforms and further provide feedback with regard to the charging and sterilization processes to which the battery is subjected.

Further, the laser welding assembly of the battery lid 60 to the underlying housing 66 eliminates the need to use fasteners to accomplish this attachment. Weld joint 221 formed by this process likewise eliminates the need to provide a separate seal to form an air-tight hermetic barrier between these components.

C. Tool/Battery Communication and Management

As depicted by FIG. 26, in a system 520 of this invention, battery 40 is used to both energize a cordless powered surgical tool 522 and provide data regarding the operational state of the tool. The depicted tool 522 is a surgical sagittal saw. It should, of course, be recognized that the system of this invention is not limited to this type of tool or to tools with motors. In FIG. 26, tool 522 is enclosed within a dashed box 632. Box 632 represents the operating room or other environment in which the tool 522 and battery 40 are used. Typically, the room in which the tool and battery are used is, to the extent possible, a sterile environment.

System 40 of this invention also includes a communications network that is constructed out of separate buses 634 and 650. One bus, bus 634, is located in the operating room. Attached to bus 634 are the components that, in real time, need to communicate with each other in order to facilitate the performance of the medical/surgical procedure. In one version of the invention, data are exchanged over bus 634 using the FireWire (IEEE 1394) protocol. It should be understood that the invention is not limited to systems using this bus protocol and that the bus protocol is not part of this invention.

In the illustrated version of the invention, a control console 636 for corded surgical tools and a navigation system 638 are two of the components attached to bus 634. Console 636 supplies power to one or more corded, electrically energized powered surgical tools that are used in the procedure (corded tools not illustrated and not part of this invention.) Navigation system 638 is used to monitor the position of the corded surgical tools relative to the body site at which the medical/surgical procedure is being performed. In some versions of the invention, navigation system 638 determines whether or not the corded tool is approaching a position at the surgical site where its use is not required. If the tool so approaches such a position, navigation system 638 sends a message reporting this information over bus 634 to console 636. Console 636, upon receipt of this invention, reduces or even negates the application of power to the corded surgical tool.

Also attached to bus 634 and located in the operating room 50 is a personal computer 640. Computer 640 serves as an input-output interface to components connected to bus 634 that might not have such interfaces. Such components, for example, include room lights and heating/cooling equipment that regulate room temperature. As is apparent from the discussion below, computer 634 also serves as the terminal through which medical personnel can retrieve information from databases outside of the operating room. Such information includes, for example, radiographic and MRI images. By entering commands into the computer 640, the medical personnel are able to present these images on displays in the operating room (displays not illustrated).

Computer 640 also serves as the bridge to the second bus, bus 650. Bus 650 is often the hospital LAN bus. Many, but not all hospitals employ Ethernet buses as their LAN buses. The exact structure of bus 650 is not relevant to the structure of this invention. Attached to bus 650 are the servers in the hospital that support the performance of the procedure and assist in monitoring the condition of the patient but that are not required in the operating room, in the sterile environment. On such server is inventory server 652. The inventory server 652 contains databases regarding the availability of tools, cutting accessories, batteries and other material used in the hospital. If a particular item is an expendable item, the inventory server 652 determines if the stock for the item has fallen below a set level. When this event occurs, inventory server 652 generates an appropriate message indicating that the item needs to be reordered.

A patient records server 654 is also attached to bus 650. Server 654 maintains a database of records associated with the patient's stay in the hospital and other data associated with the patient's medical condition and treatment. A billing server 656 creates, updates and stores records associated with the charges and payments related to the patient's stay, treatment and diagnosis. Charger 42 is also connected to bus 650.

Still another component connected to LAN bus 60 is a device monitor 658. Device monitor 658, as discussed below, based on the data retrieved from a battery 44, evaluates the state of the battery and the tool 522 the battery was employed to energize.

Another component attached to LAN bus 60 is an Internet bridge 660. Internet bridge 660 functions as the interface components between the other components connected to bus 650 and the Internet. Data output by the components attached to bus 650 are transmitted to facilities external to the hospital over bridge 660. Similarly, data from source facilities external to the hospital are supplied to the servers attached to bus 650 over bridge 660.

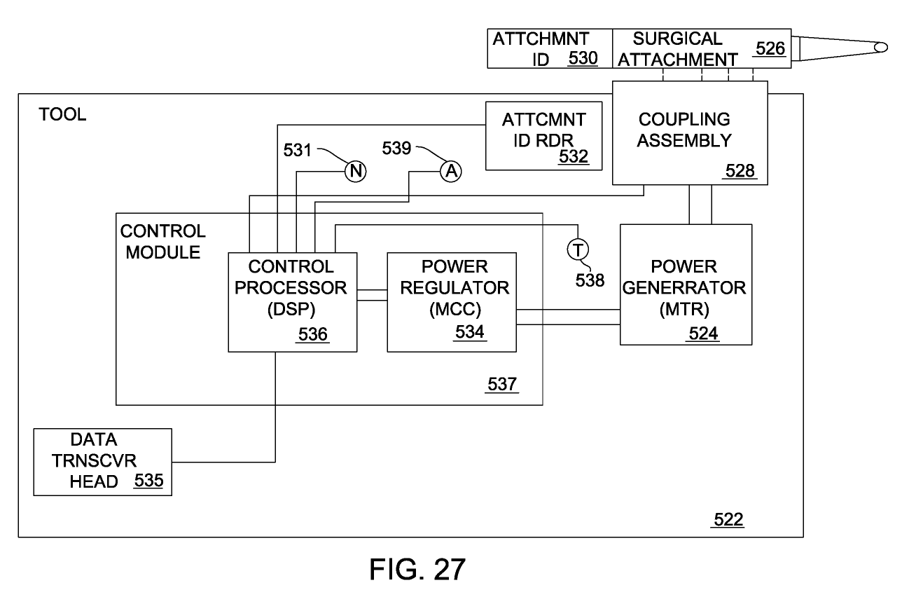

FIG. 27 is a block diagram of components of tool 522 relevant to system 520 of this invention. Tool 522 has a power generator 524. The power generator 524 is the component internal tool 522 that actuates a surgical attachment 526. In the depicted invention, the power generator 524 is a motor; surgical accessory 526 is a saw blade. A coupling assembly 528 removably holds the surgical attachment to the tool 522. Integral with the attachment is identification component 530, such as an RFID. An attachment reader 532, part of tool 522, reads the data stored by the identification component 530.

A power regulator 534 selectively applies the energy output by battery 40 to the power generator 524. The power regulator 534 applies power to the power generator 524 based on instructions received from a control processor 536. Control processor 536 generates instructions to the power regulator 534 in part based on the depression of one or more control members integral with the tool; (control member not identified). Control processor 536 receives from the attachment reader 532 the data read from the attachment identification on component 530. In FIG. 27, the power regulator 534 and control processor 536 are shown within a control module 537. This module is a sealed container within the tool 522. This invention is independent of the presence of this module.

Also internal the tool 522 is one or more sensors that monitor the operation of the tool. For simplicity only three sensors, a noise sensor 531, a temperature sensor 538 and an accelerometer 539, are illustrated. When tool 522 includes a motor as the power generating unit, temperature sensor 538 is often placed in close proximity to a bearing assembly integral with the motor. The output signal generated by temperature sensor 538 is applied to tool control processor 536. The reason for providing the tool with an accelerometer 539 is discussed below.

Tool 522 also has a data transceiver head 535. Head 535, which may be implemented in hardware and/or software, is designed to communicate with battery microcontroller 46. In one version of the invention, data transceiver head 535 consists of a software executed by tool controller 536 to exchange signals with battery microcontroller 46 and a contact integral with the tool 522 designed to establish a conductive connection with battery contact 72.

A more detailed description of the structure of a tool 522 integral with system 520 of this invention is found in the Applicants' Assignee's U.S. patent application Ser. No. 11/472,012, POWERED SURGICAL TOOL WITH SEALED CONTROL MODULE THAT CONTAINS A SENSOR FOR REMOTELY MONITORING THE TOOL POWER GENERATING UNIT, filed 21 Jun. 2005, U.S. Patent Pub. No. 20070085496 A1, now U.S. Pat. No. ______, the contents of which are incorporated herein by reference.

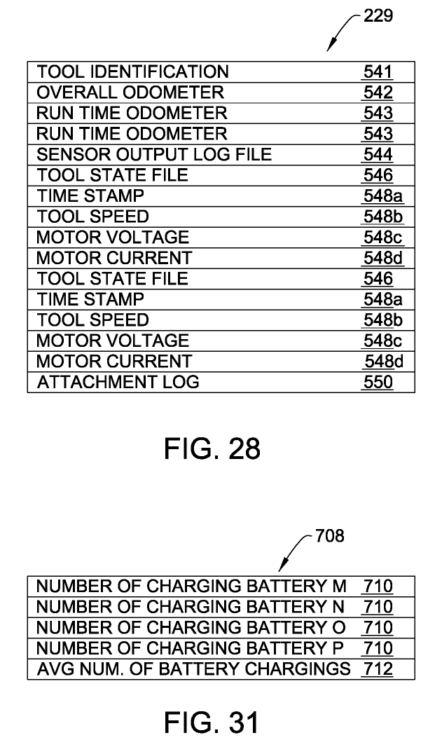

During the use of tool 522 and battery 40 of this invention, data regarding the use of the tool are stored in the battery memory 187 (FIG. 14). More particularly, these data are stored in memory tool history file 229. FIG. 28 depicts in detail types of data stored in the tool history file 229. A first file internal to file 229 is a tool identification file 541. File 541 contains data that identifies the tool 522 to which the battery 40 is attached.

Data regarding the total time the tool is run are contained in an overall run time odometer field 542. Data indicating the times the power generator 524 is run above or below specific operating state(s) is stored in one or more operating mode run time odometer fields 543 (two shown in FIG. 28). For example, if the tool power generator 524 is a motor, a first field 543 may store data indicating the overall time the motor is run at or above a particular speed. A second field 543 stores data indicating the overall time the motor is run under load. Tool control processor 536 makes a determination of whether or not the motor is run under load based on the current drawn by the motor. If the tool power generator 524 is a part of an ablation tool, the overall odometer field stores data indicating the overall time the tool is actuated; a run time odometer field is used to store data indicating the time the tool or its attached accessory is heated to a particular temperature.

Tool history file 229 also contains a sensor output log file 544. File 544 is used to store data based on the signals generated by the sensors internal to the tool 522. In some versions of the invention, the data stored in file 544 are signals representative of the actual parameter sensed by the sensor. For example, if one sensor is a noise sensor 531, the data in file 544 can include data indicating if noise above a certain threshold level was exceeded and/or the time it was so exceeded. If a temperature sensor 538 is present, the data in file 544 can include data indicating the peak temperature detected by the sensor.

File 544 may also includes flags that are set as a function of the tool or environmental states sensed by the sensors. Thus, system 520 of this invention is set so that if the sensor 538 detects a temperature above a threshold level, a flag indicating that the tool reached such a temperature is set. The accelerometer 539 is used to evaluate whether or not the tool is dropped. (The sudden acceleration of the tool at 9.8 m/s² followed by a rapid deceleration to a speed of 0 m/s is indicative of the tool being dropped). Accordingly, file 544 may include a flag field that is set if control processor 536, based on the output of the sensor signal, determines that the tool has experienced such an acceleration and/or deceleration profile. Some tools are configured so that the power regulator 534 and control processor 536 are capable of, in response to a trigger event, limiting the current applied to the power generator 524. In this type of tool, the file 544 also contains a field in which data are stored regarding the number of times the current is so limited.

Also internal to output log file 544 are a number of tool history state record files 546. In FIG. 28 only two state record files 546 are shown. Each state record file 546 contains data that, collectively, provide an indication of the operating state of the tool when the power generator 524 is actuated. For example, when the tool includes a motor, each state record file 546 includes a time stamp field 548a. Field 548a includes an indication of when the data in the particular file was obtained. This time stamp may contain a real time scalar value. Alternatively, the time stamp contains a cumulative time scalar value starting from a trigger event. One such trigger event can be the attachment of the battery 420 to the tool 522. If the power consuming unit is a motor, other fields in file 546 contain indications of: tool speed, field 548b; voltage across the motor rotor, field 548c; and current through the motor 548d. If the tool is some sort of ablation tool, one of the fields in file 546 indicates the temperature of tool or the attached accessory.

Also internal to tool history file 229 is an attachment log file 550. Accessory log file 550 contains data that identifies the specific attachment(s) 526 attached to the tool 520. These data are based on the data collected by the tool attachment reader 532. In some versions of the invention, each attachment log file contains for each attachment, total run time odometer data, operating mode run time data and data based on the output from the sensors during use of the attachment.

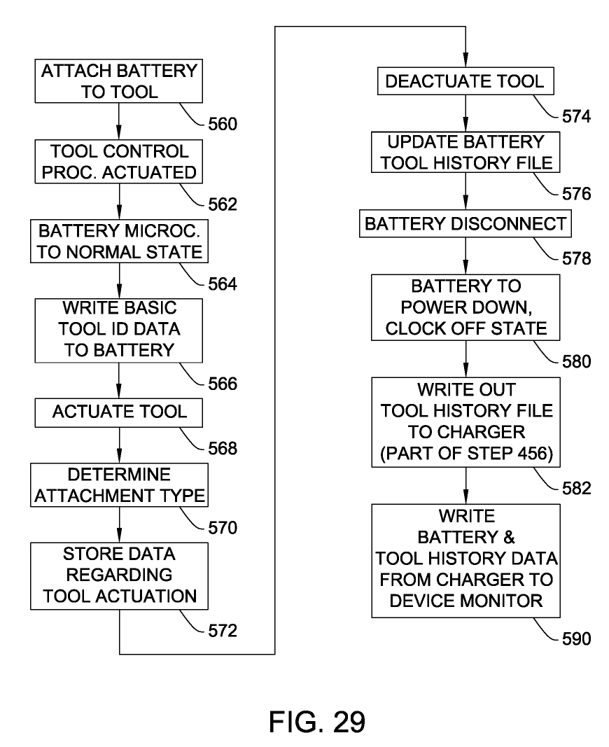

A process by which data are loaded into and retrieved from the battery microcontroller memory 187 is now described by reference to FIG. 29. Step 560 is the coupling of the battery to the tool 522. As a result of this step, there is immediate current flow to the tool and the subsequent actuation of the tool control processor 536, step 562. As part of the initial actuation sequence, tool control processor 536 pulls the one-wire communication line internal to the battery low so as to cause battery microcontroller 46 to transition from the power down, clock off state to the normal state, step 564.

Tool control processor 536, in a step 566, then writes into battery microcontroller memory file 187 data identifying the tool. At this time, tool controller also writes into the battery initial voltage field 227, data indicating the initial voltage of the battery. This voltage is determined by the control processor 536. In some versions of the invention, the voltage across the battery or a divided down representation of this potential is applied to an analog to digital converter (ADC) connected to the control processor 536, (ADC not illustrated). In some versions of this invention, this ADC is integral with control processor 536. The digitized representation of battery voltage produced by this circuit/component is the data that are stored first in field 227 and then in field 228.

Step 568 represents the actuation of the tool. At this time, tool control processor 536 engages in an initial collection of data regarding the operation of the tool, step 570. Step 570 involves determining from the attachment reader 532 the identity of the specific attachment 526 coupled to the tool. The data obtained in step 570, as part of the step, are stored in a RAM associated with the tool control processor 536 (RAM not shown).

As long as the tool continues to be actuated, tool control processor 536, in a step 572 acquires and stores data regarding the tool actuation. These data, for example, include total run time odometer data and data indicating run time in one or more states, for example, speed level, running at load or operating at a particular temperature. These data further include, at various times, the tool speed, motor voltage and current through the motor. In some versions of the invention, these parameters are recorded at least once a second. In more preferred versions of the invention, these data are recorded at least once every 0.1 seconds. These data are likewise stored in the microcontroller RAM.

In a step 574 the tool is deactuated. In an immediate next step 576, tool control processor 536, through data transceiver head 535, updates the data log of the use of the tool in the battery microcontroller memory tool file 229. Thus, after each individual actuation of the tool, the data recorded in the sensor output log file 544 are updated. This updating also includes writing to battery memory field 228 data indicating the voltage of the battery immediately upon deactivation of the tool power generating unit. This is the voltage produced by the ADC integral with the tool to which the battery voltage or its divided down equivalent is applied. The updating also includes writing additional tool state files 546 to the tool to form a log of the activation of the tool. The attachment log 550 is also updated to include data identifying the type of attachment 530 used in the actuation.

It should be appreciated that not all of the data may be updated. For example, if peak temperature is measured during the first actuation of the tool, the temperatures reached in any subsequent actuations are not recorded.

Once use of the tool 522 is completed, battery 40 is disconnected, step 578. This results in battery one-wire communication line going high. This transition is detected by the interrupt circuit internal to the microcontroller 46. This signal staying high for an extended period of time is interpreted by the microcontroller as indicating the battery has been disconnected from the tool 522. Therefore, in a step 580, the microcontroller returns the battery to the low power consuming, power down, clock off state.

At the conclusion of the procedure, the battery 40 is typically removed from the operating room 632 and sterilized. After sterilization, the battery is placed in charger 42. (The steps of sterilizing the battery and placing it in the charger 42 not shown.)

As discussed above with respect to FIG. 24A, once the battery 40 is attached to the charger 42, in step 456, the data in the battery microcontroller memory 187 are written out to the charger main processor 298. As part of this process, the data in the tool history file 229 are read out, step 582 of FIG. 29.

Charger main processor 298, in turn, forwards the data retrieved from the battery memory 187 over bus 650 to device monitor 658, step 590. Device monitor 658, then reviews these data to determine if it indicates that either the battery 40 or tool 522 are in or approaching a state wherein maintenance is desirable/required.

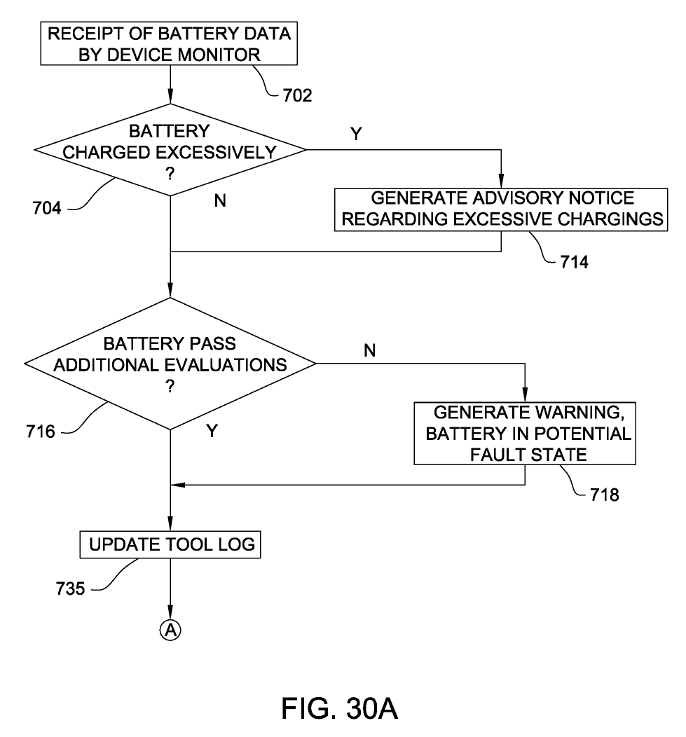

Exemplary evaluations of the battery and tool evaluation processes performed by device monitor 658 are now described with regard to the flow charts of FIGS. 30A-C. Device monitor 658 can be a general purpose processor programmed to perform the below described evaluations. Also integral with the device monitor 658 is a memory for storing the evaluation instructions, the data written to the device manager and the data generated by the device manager processor. Step 702 represents the initial receipt of the battery and tool history data.

In one test, represented by step 704, device monitor 658, determines if the battery 40 has, in comparison to similar batteries, been subjected to excessive use. Such use can occur if personnel, out of habit, repeatedly use one or two batteries out of larger stock of available batteries.

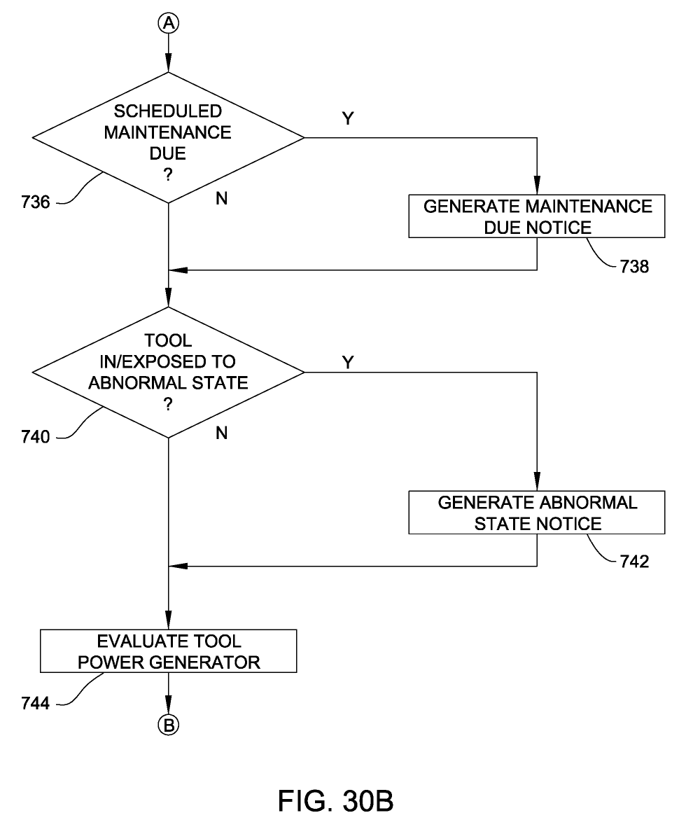

To perform this evaluation, device monitor 658, based on the data initially contained in the number of chargings field 197, compares the number of times that particular battery has been charged to reference number. This reference number can be an average of the number of times a particular set of batteries in the hospital have been charged. This average value can be obtained by the device monitor 658 maintaining, for each battery in the hospital, at least a partial duplicate of the data contained in the battery memory 187. A file 708 representative of these data is illustrated by FIG. 31. At a minimum, these data include for each battery an identifying code and the number of chargings, fields 710 of file 708. Based on the number of chargings for the individual batteries, device monitor 658 determines the average number of chargings, step not shown. This average, stored in field 712 of file 708, is the value against which the total number of chargings the battery 40 under evaluation is compared.

If, in step 704 device it is determined that the battery under evaluation has been in comparison to other batteries, excessively charged, device monitor 658 generates one or more advisory notices, step 714. An advisory notice can take the form of an alphanumeric message that is forwarded over bus 650 to person charged with maintenance of the hospital equipment. The notice may be forwarded to persons outside the hospital over the Internet bridge 660.

In a step 716, device monitor 658 conducts additional evaluations to determine whether or not the battery 40 can be used in a subsequent procedure. It should be understood that the evaluation of step 716 includes one or more individual evaluations to determine whether or not the battery is or is approaching a state in which it may malfunction. For example, some batteries, those with Li-ion cells, have internal “gas gauges.” A battery gas gauge provides a measure of the charge in the battery. Post charging data representative of this value are written into the memory 187; the field into which these data are written, not shown. Device monitor 658, in step 716 reviews these data and compares the measured charge level to a target level. If the stored charge is less than the target level, in a step 718, device monitor 658 generates a warning announcing that the battery 40 is in this condition.

Also in step 716, device monitor 658 reviews the initial and final voltages out of the battery from fields 227 and 228, respectively. These data are reviewed to determine if the battery, while in use, output voltages within the target range. As part of this evaluation, in step 716, may also evaluate output voltage with regard to the tool run times from the odometer fields 542 and 543. In this version of the evaluation, the final voltage, or the difference between the initial and final voltages, is compared to a reference voltage level (voltage difference) that is function of the overall time the tool was run and/or run under load. Thus, if the odometer data indicates that, in the last use, the tool was run for a relatively short time, the reference voltage against which the final voltage is compared would be relatively high (the difference between initial and final battery voltages would be relatively small.) Alternatively, if the odometer data indicates that the tool was run for a relatively long amount of time, the device monitor would generate a final reference voltage value that is relatively low, (the difference between the initial and final battery voltages would be relatively high.)

If this sub-evaluation of step 716 indicates that the battery 40, in its last use, underwent an out of normal range voltage drop, step 718 is executed. This gives hospital personnel knowledge of the battery's performance so steps can be taken to prevent its reuse.

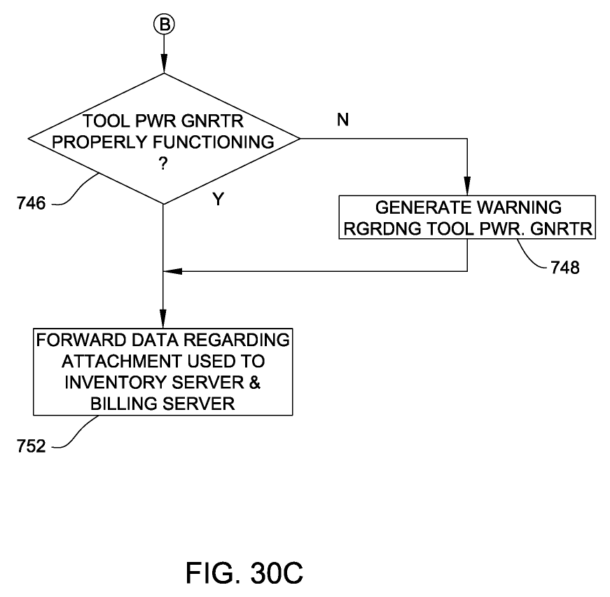

Device monitor 658 also evaluates the condition of the tool 522 based on the data read from the tool history file. For each tool 522, a use log is maintained. This log may be maintained by any component connected to the network, for example, either the device monitor 658 or the inventory server 652. FIG. 32 illustrates some of the data that are maintained in a log 720 for a specific tool. The data in the log 720 is analogous to the data in the tool history file 229 stored after a single use of the tool. There is a tool identification field 722, an overall odometer field 724 one or more run time odometer fields 726, a sensor log output file 726 and one or more tool state files 730. The odometer fields 724 and 726 include data indicating overall elapsed time the tool is used. Some of the fields may contain information that are slightly different from the information stored in a per-use tool history file 229.

Thus, one run time odometer field 726 may contain data indicating many hours the tool has been used since it was subject to maintenance and/or an overhaul.

The sensor run time file 728, contains more than data indicating the output signals from certain sensors. File 728 also includes time stamps that allow reviewing personnel to determine when particular sensed events occurred. The times of these time stamps may be generated based on time stamps added to the data when downloaded by the charger processor 298. Tool state files 730 contain data that, for a number of separate times the tool was used, describe various characteristics of the tool state. These characteristics include, tool speed, voltage across the power generating unit, current drawn and accessory attached.

In a step 735, device monitor 658 updates the tool log 720 for the tool 522 to which the battery 42 was connected. In step 735, the data in the tool history file 229 for the battery the charger 44 forwarded to the device monitor 658 are used to update the tool log 720. Implicit in this is the process of adding the time stamps to these data. For example, the odometer data reflecting the overall time and the time under load for the last use of the tool are added into the time records previously stored overall odometer field 724 and the appropriate run time odometer field 726.

Also in step 735, records of any unusual events, such as the sensing of excessive temperature, noise or acceleration/deceleration are written into the sensor output log file 728. The tool state data from the files 546 are written into the tool log 720 as tool state files 730.

Device monitor then conducts a number of evaluations to determine if the tool is performing properly or the tool is in a state wherein maintenance is desirable. A first one of these evaluations, is the scheduled maintenance needed evaluation 738. In evaluation 738, device monitor 658 compares the data in the log overall odometer field 724 in an appropriate one of the run time odometer fields 726 to a reference run time value. This reference run time value is a time close or equal to that a run time value at which the tool should be subjected to scheduled maintenance.

If, as a result of the execution of the evaluation of step 736, it is determined that the tools run time is approaching or at that which scheduled maintenance is needed, device monitor 658 outputs an appropriate notice, step 738. Typically this notice is forwarded to maintenance station. Most hospitals do not support their own maintenance stations for this type of work. Accordingly, the notice is forwarded over Internet bridge 660 to maintenance station that is remote from the hospital. Receipt of this notice serves as the cue to the personnel at this station that they should obtain the tool from the hospital for the scheduled maintenance. As part of this process, a loaner version of the tool can be made available.

Thus, this feature of the system of this invention makes it possible for a tool to be submitted for maintenance/repair and a loaner unit provided before the need to perform such maintenance/repair is overdue. Another aspect of this feature of the invention, is that by providing the loaner tool when requesting return of the hospital's tool for maintenance, the system ensures that the even when the hospital's tool is recalled for maintenance, the substitute tool is available for use.

In a step 740, device monitor reviews the data in the output log file 544. In this review, the data are analyzed to determine if they indicate the tool is in or could be in an abnormal state. Thus, the temperature data is reviewed to determine if, during operation, the tool 522 reached an abnormally high temperature. The accelerometer data are reviewed to determine if it indicates the tool may have been exposed to an appreciable mechanical shock. If the tool has a noise sensor 531, the noise sensor data are reviewed to determine if the tool generated an abnormally high level of noise.

If the results of any one, or a particular combination of these reviews are positive, a warning message is output, step 742. This warning message is output over bus 650. The warning message may be sent to hospital personnel responsible for the maintenance of the tool. Alternatively, or in addition to informing hospital personnel, the message may be output over Internet bridge 660 to the outside maintenance station. The recipient of the message, either internal or external to facility at which the tool is used, may, based on the nature of the warning, decide it is necessary to arrange maintenance of the tool so as to avoid the occurrence of a fault later during the performance of a procedure. This information could also be used by its recipient to serve as a basis for advising the medical personnel that a fault could occur or that further care of the tool is needed.

In a step 744, device monitor 658 evaluates the condition of the tool power generator 524. This monitoring is performed by reviewing the data in the tool state files 730. This review may be just of the data in the tool state files 730 for the last use of the tool 522. Alternatively, this review may further be base on tool state data for a number of past uses of the tool 522. In the evaluation of step 744, the device monitor 658 compares the data from the tool state files to reference level data. For example, some tool state files report data indicating for when the motor is run in a no load state, the high speed state. The current drawn by the motor when in this state is reviewed. This current level is compared to a reference current level. If the no load current draw of the motor is above the reference current level, it may be an indication that the motor is underperforming.

As a result of the evaluations of step 744, device monitor 658, in step 746 determines if the tool power generator 524 appears to be functioning properly. In the event the evaluations and testing of steps 744 and 746 indicates there is potential question regarding the performance of the power generator 524, device monitor 658 generates an appropriate warning, step 748. This warning, which may go to a recipient internal and/or external to facility in which the tool 522 is output to indicate that the tool may be or is in a state in which maintenance is required to avoid malfunction.

Device monitor 658 also assists in the inventorying of the surgical attachments used with the tool. As discussed above, in the event the tool is capable of determine the type of attached attachment, data reflecting these information are stored in attachment log 550 of the tool history file 229. Device monitor 658, upon receiving these data, forwards the data to the inventory server and billing server, step 752. The inventory server 652 uses these data to update the stock level of the attachment. Billing server 656 uses these data to ensure that the charges with regard to the use of the attachment and tool on the patient are properly noted.

Thus, even though the battery 40 and tool 522 of the system of this invention are cordless, data regarding battery and tool use are available to persons responsible for ensuring their availability.

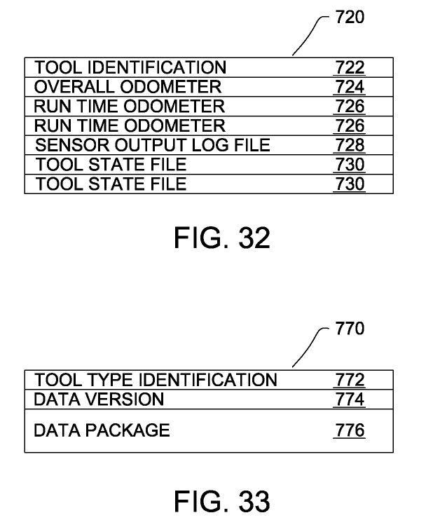

The system of this invention not only uses the battery 40 to transfer data from the tool 522 to the external network, the battery is also used to transfer data and instructions to the tool. Battery memory 187 may contain one or more tool upload data files 770, one of which is seen in FIG. 33. Each tool upload data file 770 contains a tool type identification field 772. Field 772 contains data indicating the type of tool into which the contents of file 770 are to be loaded. File 770 also contains a version field 774. Field 774 contains data indicating the version number of the data for the tool contained in the file. The tool upload data file 770 also contains an upload data package 776. Package 776 contains the data that are actually to be uploaded into the tool 522. While package 776 is described as a “data” package, typically, the actual contents of this package are revised sets of operating instructions for the control processor 536 internal to the tool 522.

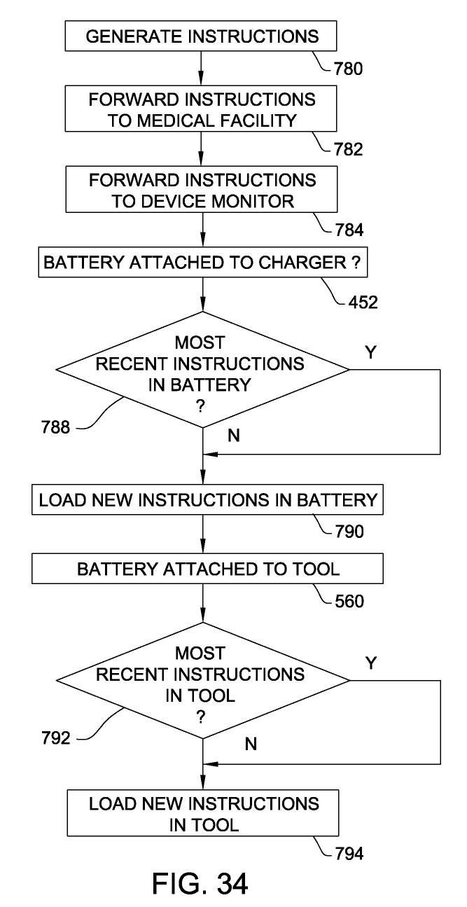

As represented by step 780 of FIG. 34, the process of uploading data (operating instructions) into the tool starts with the generation of the instructions. Typically, this step occurs at the location where the tool is manufactured or the maintenance station. Step 782 represents the transmission of the assembled data file 770 to the medical facility at which the tool is used. The Internet bridge 660 at the facility then forwards the instructions to the device monitor 658, step 784.

Once the instructions are stored in the device monitor 658, they are ready for loading into the individual batteries. When the battery 40 is placed in the charger 42 one or more tool upload data files 770 are loaded into the battery memory 187. Chronologically, this process may occur after step 590, (FIG. 29) the downloading of the data stored in the battery regarding its last operation.

In one version of the invention, after step 590, the downloading of the data in the battery memory, the device monitor, through the charger 42, queries the battery to determine, if for each tool 522 with which the battery is used contains the most recent instructions (data) for the tool. This determination, step 788, is made by comparing the version number in field 774 for the data file 770 for a tool with the version number for the most recently stored instructions. If the numbers match, then the battery memory 187 already contains the most recently generated instructions.

If the evaluation of step 788 indicates that the device monitor 658 contains a more recent set of instructions, these instructions are uploaded to the battery through the charger, step 790.

When needed to charge a tool, battery 42 is coupled to the tool 522, step 560. Tool control processor 536 scans the tool upload data files 770 stored in the battery, step not shown. By reviewing the contents of the tool identification fields 772, control processor 536 identifies the upload data file 770 for that specific tool. The tool control processor 536, in a step 792, compares the version number of the file 770 in the battery memory 187 with the version of the data (instructions) stored in the processor's internal memory. If these numbers match, the data (instructions) in the file data package 776 are not uploaded.

The evaluation of step 792 may reveal that the version of the data in the file 770 stored in the battery is later than the version of the data presently stored by the processor. In this event, the tool control processor reads the data out of the file data package 776 and writes the data into the processor's internal memory, step 794. Tool control processor 532 then regulates operation of the tool based on the updated instructions obtained from the battery 40.

Thus, the system of this invention is further designed to upload into cordless tools 522 packages of updated instructions for regulating the operation of the tools.

### V. Alternative Embodiments

It should be appreciated that the foregoing description is directed to one specific version of the battery and related components of the system of this invention. Other versions of this invention may have alternative features, constructions and methods of execution.

Thus, there is no requirement that each of the above inventive features be found in all embodiments of the invention.

For example, in some versions of the invention, the battery may not be sealed from the ambient environment. In these and other versions of the invention, the sensor internal to the battery may be one that is used to determine the exposure to an environmental agent other than temperature that could adversely affect charge storage by the cells 44. Thus, the sensor internal to the battery could detect humidity. If the sensor detects that the atmosphere within the battery is of relatively high humidity, data logging this event are stored in the battery memory. Another alternative sensor is an accelerometer. This sensor, like the accelerometer internal to the tool 522, is used to monitor if the battery is dropped.

Again, such an event is logged in the battery memory. Then if the charger 42, upon reading the stored data, recognizes that the battery was exposed to the unusual environment event, the charger would subject the batter to the complete state-of-health evaluation.

Alternatively, an accelerometer or other sensor may be employed to sense whether or not the battery is excessively vibrated. Data regarding the excessive vibration is likewise stored in the battery memory.

With regard to the above it should also be understood that occurrence of one of the above environmental events may be the trigger that causes the battery to transition from the power down mode to the normal mode.

Further it should be appreciated plural such environmental sensor may be fitted to the battery.

Similarly, alternative constructions that come within the scope of the invention are also possible. Thus, a battery may be provided with cells having less or more than the eight (8) cells illustrated in the version of the invention illustrated in FIG. 6. For example, to provide a battery with ten (10) cells that has the heat dissipating cell arrangement of this invention, plural middle rows of cells, each having no more than two (2) cells per row may be provided. Also outer rows of cells with fewer or more than the three (3) cells may be provided depending on the number of cells the array is to have. In some versions of the invention, arrays of cells may be stacked one on top of the other.

Similarly, there is no requirement that in all versions of the invention the laser welding be performed using a laser that emits photonic energy at 980 nanometers. For example, in some versions of the invention, the laser welding may be performed with a laser that emits coherent light energy at 808 nanometers. Again, this is just exemplary, not limiting. It should likewise be appreciated that other medical equipment, not just batteries, may be laser welded using the process of this invention.

In this vein, it is further understood that there is no requirement that in all versions of the invention, the top of the housing always function as the component that is seated in the base and heated by the photonic energy. In other versions of the invention, this relationship may be reversed. Clearly, the laser welding may be used to assemble other components forming the housing together. Thus, the method may be used to secure multiple panels together.

Likewise, there is no requirement that the geometries along which the components forming the battery housing meet have the disclosed geometry. In some versions of the invention, either neither or only one of the surfaces along which the weld seam is formed may have a tapered profile.

Similarly, in some versions of the invention, the battery may only contain a non-volatile memory. When the battery is attached to the tool, the tool writes data to the memory. Then, when the battery is attached to the charger the charger reads out the data written into the memory by the tool so the data can be forward to the appropriate destination.

Clearly, there is no requirement that all versions of the invention be constructed to energize and communicate with powered surgical tools. Thus, the battery of this invention can be used to energize power consuming devices other than surgical tools. The communications system of this invention can be used to obtain data from devices other than cordless surgical tools.

It should likewise be appreciated that the components and process steps of this description are only exemplary and not limiting. For example, in some versions of the invention, the multiple components internal to the battery may function as the memory in which data are stored and the device that writes to and reads data from the memory. Likewise, in some versions of the invention, tool control processor 536 may, during actuation, simultaneously log data into the battery memory.

Circuit variations are also possible. Thus, in some versions of this invention, the end of resistor 209 opposite the V_{TEMP}_{_{—}}_{REF} junction may be tied to the output pin of voltage converter 182. In these versions of this invention, the end of resistor 210 opposite V_{TEMP}_{_{—}}_{REF} junction is tied to V_{SS} or the BATT− terminal. An advantage of this version of the invention is that it results in a V_{TEMP}_{_{—}}_{REF} signal that does not vary with manufacturing differences in microcontroller 46.

There is no requirement that all chargers of this invention be able to simultaneously charge plural batteries. There is no requirement a charger accept different modules so the charger is able to charger different types of batteries.

Also, it should be recognized that the power generator 524 need not always be a motor. The power generator may be a device that generates electrical energy, RF energy, ultrasonic energy, thermal energy or photonic energy.

Returning to FIG. 12, it can be seen that the battery may also be provided with a wireless transceiver 602. This transceiver may be an RF or IR unit. In some versions of the invention, the transceiver may be a Bluetooth transceiver. When the battery is connected to the tool, transceiver 602 exchanges signals with a complementary transceiver 604 attached to bus 586. Thus, this version of the invention allows real time communication between the cordless tool 522 and other operating room equipment through battery 40. For example using this arrangement, a voice actuated control head 606 can be used to regulate tool actuation. Thus, a command entered through control head 606 is packetized and sent over bus 586 to transceiver 604. Transceiver 604 broadcasts the command to battery transceiver 602. The command is transferred from the battery transceiver to the battery microcontroller 46. Microcontroller 46, in turn, forwards the command through the tool transceiver head 535 to the tool processor 530. Tool processor 530, in turn, generates the appropriate commands to the power regulator 534 to cause desired actuation of the power generator 524.

Similarly, surgical navigation system 638 may likewise be connected to the tool through transceivers 602 and 604. The surgical navigation system tracks the position of the tool 520 and attachment 526 relative to the surgical site to which the attachment is applied. If the navigation system determines that the attachment is being position at a location at which it should not be used, the attachment would generate a stop command. This command is transmitted through transceiver 604 to transceiver 602 and, from transceiver 602, to the tool control processor 536. Tool control processor 536, upon receipt of the command, at least temporarily deactivates or slows operation of the tool 522.

It should likewise be understood that the not all batteries of this invention may be designed to withstand the rigors of sterilization. Alternatively, the features of this invention may be incorporated into an aseptic battery pack. this type of battery pack include a sterilizable housing that defines a void space for receiving a removable cell cluster. A sealable lid associated with the housing allows insertion and removal of the cell cluster. With this battery pack, prior to sterilization, the cell cluster is removed from the housing. Thus the cells of an aseptic battery pack are spared the rigors of autoclave sterilization. The Applicants' Assignee's U.S. patent application Ser. No. 11/341,064, filed 27 Jan. 2006, ASEPTIC BATTERY WITH REMOVABLE CELL CLUSTER, U.S. patent Pub. Ser. No., ______, now ______, the contents of which are incorporated herein by reference, discloses one such aseptic battery pack. Still, the features of this invention may be built into the housing and or cell cluster of an aseptic battery pack.

The actual physical components of the system may also be different from what has been described. For example, in some versions of the invention, the device monitor 658 is integral with the charger 42. Thus, in these versions of the invention, either the charger processor 298 or a separate processor internal to the charger 42, receives the data regarding the operation of the battery and tool 522, and based on these data, evaluates the states of these devices. An advantage of this construction of the system is that the need for a separate hardware component, a stand alone device monitor, is eliminated.

Likewise, the battery processor 185, not the tool processor, may regulate the uploading of new instructions into the tool, the process of FIG. 34.

Likewise, there is no requirement that in all versions of the invention, the device monitor 658 that evaluates the operational state of the battery 40 and tool 522 be in the hospital. In some versions of the invention, this component may be part of a server that is at a repair station remote from the hospital. When the battery is placed in the charger, the charger processor outputs the data retrieved from the battery memory and outputs over the bus 650. The data are output with a destination instructions indicating it is to be sent to the remote device monitor 658. Internet bridge 660, upon receipt of these instructions and the associated data, forwards the data to the device monitor 658. An advantage of this arrangement is that a single device monitor can evaluate the operational states of equipment used at a number of different hospitals.

Similarly, the various processes may be different from what has been disclosed. For example, in some versions of the invention, the data (instructions) that are to be written into the tool 522 may be stored in charger 42. Then the steps associated with written these data into the battery memory are performed by the charger 42.

Further, it should be recognized that the described evaluations the device monitor 658 performs to determine whether or not the battery 40 or tool 522 are properly functioning are only exemplary. The actual evaluations performed on the tool or battery may be different from what has been described.

Similarly, it should be appreciated that this invention is not limited to systems wherein a one-wire protocol is used to write data to/read data from the battery memory 187.

Likewise, in some versions of the invention, a unit other than the battery may be used to retrieve data from the cordless tool 522 and load data into the tool. Thus, in some versions of the invention the device monitor 658 may include a receiving head. This receiving head is structurally similar to the head of the battery so that a tool can be fitted to the head. The head has contacts that are used to establish an electrical connection to the memory integral with the tool control processor 536. At some point of time between uses of the tool 522, the tool is coupled to the manager. The data in the tool memory, which is similar to the tool data stored in the battery memory, are read out to the device monitor. Revised instructions for regulating tool operation are also directly loaded into the tool from the device monitor through this head. This construction of the invention thus eliminates the need to provide the battery with sufficient memory for storing all the data that may be downloaded to it from the tool 522.

Thus, it is an object of the appended claims to cover all such variations and modifications that come within the true spirit and scope of this invention.

What is claimed is:

1. A powered surgical tool system, said system including:
a powered surgical tool having: a power generating unit; a surgical attachment for performing a surgical/medical procedure that is actuated by said power generating unit; a control circuit for regulating actuation of said power generating unit; and a data transmission head that is connected to said control circuit wherein, said control circuit is further configured to output data regarding the operation of said powered surgical tool through said data transmission head;
a battery that is removably attachable to said powered surgical tool, said battery having at least one rechargeable cell for energizing said tool power generating unit and a memory that is removably connected to said tool data transmission head and that is configured to store data received from said data transmission head regarding the operation of said tool; and
a static unit for removably receiving said battery, said static unit having a device for reading from the data in said battery memory, including the stored data regarding operation of said powered surgical tool.

2. The powered surgical tool system of claim 1, further including a charger for removably receiving said battery, said charger including a current source for applying a charging current to said at least one rechargeable cell, and said charger is said static unit for reading the data in the battery memory.

3. The powered surgical tool system of claim 1, wherein said static unit is connected to a communications bus and is configured to output over the communications bus the data read from data stored regarding operation of said powered surgical tool.

4. The powered surgical tool system of claim 1, further including a device monitor, said device monitor configured to: receive from said static unit the data regarding the operation of said powered surgical tool; based on the data from said powered surgical tool, evaluate the operation of the powered surgical tool; and, based on the evaluation of power operation, selectively generate data regarding the state of said powered surgical tool.

5. The powered surgical tool system of claim 1, wherein:
said powered surgical tool power generating unit is a motor, and said control circuit is able to output through said data transmission head to said battery memory data regarding at least one of: the speed of the motor; the voltage across the motor; and the current drawn by the motor; and
a device monitor receives from said static unit the data regarding the operation of said powered surgical tool and said device monitor is configured to, based on the at least one of the speed of the motor, the voltage across the motor, and the current drawn by the motor, evaluate the operation of said powered surgical tool; and based, on the evaluation of said powered surgical tool, selectively generate data regarding the state of the powered surgical tool.

6. The powered surgical tool system of claim 1, wherein:
said powered surgical tool includes at least one sensor for monitoring the operation of said powered surgical tool and said powered surgical tool control circuit is configured to: receive a signal from said at least one sensor; and output through said data transmission head to said battery memory data regarding the sensor signal; and
a device monitor receives from said static unit the data regarding the operation of said powered surgical tool and said device monitor is configured to, based on the data regarding the sensor signal, selectively generate data regarding the state of said powered surgical tool.

7. The powered surgical tool system of claim 1, wherein:
said attachment is removably attached to said powered surgical tool;
said control circuit is further configured to identify the said attachment attached to said powered surgical tool; and write into said battery memory through said data transmission head data identifying the said attachment; and
said static unit is further configured to read out of said battery memory the data identifying the said attachment.

8. A method of operating a battery powered surgical tool, said method including the steps of:
energizing a powered surgical tool with a rechargeable battery removably attached to the tool;
with the powered surgical tool, generating data regarding the operation of the tool;
storing the data regarding the operation of the operation of the powered surgical tool in a memory integral with the battery;
removing the battery from the powered surgical tool, placing the battery in a charger and charging the battery with the charger; and
writing out from the battery memory the stored data regarding the operation of the powered surgical tool and evaluating the operation of the tool based on the data regarding the operation of the powered tool.

9. The method of operating a battery powered surgical tool of claim 8, wherein, said step of writing out from the battery memory the stored data regarding the operation of the powered surgical tool is performed when the battery is attached to the charger.

10. The method of operating a battery powered surgical tool of claim 8, further including the step of, using the data regarding the operation of the powered surgical tool to evaluate the state of the powered surgical tool.

11. The method of operating a battery powered surgical tool of claim 8, wherein:
during said step of energizing the powered surgical tool, data are generated regarding the energization of the powered surgical tool;
in said step of storing data in the battery memory, the data generated regarding the energization of the powered surgical tool are stored;
after the data regarding the operation of the powered surgical tool are written out of the memory, the data generated regarding the operation of the powered surgical tool are used to evaluate the state of the powered surgical tool.

12. The method of operating a battery powered surgical tool of claim 8, wherein:
the powered surgical tool includes at least one sensor that outputs signals regarding the operation of the powered surgical tool;
in said step of generating data regarding the operation of the powered surgical tool, data are generated based on the signals output by the at least one sensor;
in said step of storing data in the battery memory, the data generated based on the signals output by the at least one sensor are stored; and
after the data regarding the operation of the powered surgical tool are written out of the memory, the data stored based on the signals output by the at least one sensor are used to evaluate the state of the powered surgical tool

13. The method of operating a battery powered surgical tool of claim 8, wherein:
a removable accessory is attached to the powered surgical tool;
during said step of energizing the powered surgical tool, actuating the cutting accessory with the powered surgical tool;
with the powered surgical tool, obtaining data describing the cutting accessory;
as part of said step of storing data regarding the operation of the powered surgical tool in the battery memory, storing data describing the cutting accessory; and
as part of said step of writing out data from the battery memory, writing out the data describing the cutting accessory.

14. The method of operating a battery powered surgical tool of claim 8, wherein:
said steps of energizing the power generating tool, storing the data regarding the operation of the powered surgical tool in the battery memory and writing out of the battery memory the data regarding the operation of the powered surgical tool are performed at a medical facility; and
after said step of writing out of the battery memory the data regarding the operation of the powered surgical tool, the data are transmitted to a facility remote from the medical facility.

15. A powered surgical tool system, said system including:
a powered surgical tool, said tool having: an attachment for performing a medical procedure; a power generating unit that actuates said attachment; a monitoring circuit for monitoring the operation of the powered surgical tool and outputting data regarding the operation of said powered surgical tool;
a battery that is removably attachable to said powered surgical tool, said battery having: at least one rechargeable cell for energizing said tool power generating unit; and a memory that is connectable to said tool monitoring circuit for receiving and storing the data regarding the operation of said powered surgical tool; and
a charger for removably receiving said battery, said charger having: a current source for charging said battery at least one rechargeable cell; and a processing circuit connectable to said battery memory for reading out of said battery memory the stored data regarding the operation of said powered surgical tool.

16. The powered surgical tool system of claim 15, further including a device monitor, said device monitor configured to: receive the data read by said charger regarding the operation of said powered surgical tool; based on the data from said powered surgical tool, evaluate the operation of said powered surgical tool; and, based on the evaluation, selectively generate data regarding the state of said powered surgical tool.

17. The powered surgical tool system of claim 15, further including a device monitor separate from said charger, said device monitor configured to: receive from said charger the data read by said charger regarding the operation of said powered surgical tool; based on the data from said powered surgical tool, evaluate the operation of said powered surgical tool; and, based on the evaluation of power operation, selectively generate data regarding the state of said powered surgical tool.

18. The powered surgical tool system of claim 15, wherein:
said tool monitoring circuit is configured to: monitor the operation of said tool power generating unit; and store in said battery memory data regarding the operation of said tool power generating unit; and
a device monitor receives the data read by said charger from said battery memory and said device monitor is configured to, based on the read data, including the data regarding the operation of said power generating unit, evaluate the operating state of said powered surgical tool.

19. The powered surgical tool system of claim 15, wherein:
said tool monitoring circuit includes at least one sensor capable of monitoring the operation of said tool, said at least one sensor outputting signals regarding the operation of said powered surgical tool and said monitoring circuit is configured to store in said battery memory data based on the signals output by said at least one sensor; and
a device monitor receives the data read by said charger from said battery memory and said device monitor is configured to, based on the read data, including the data based on the signals output by said at least one sensor, evaluate the operating state of said powered surgical tool.

20. The powered surgical tool system of claim 15, wherein:
said attachment is removably attached to said powered surgical tool;
said monitoring circuit is further configured to identify the said attachment attached to said powered surgical tool; and write into said battery memory data identifying the said attachment; and
said charger is further configured to read out of said battery the data identifying the said attachment.
